SEQUENCE LISTING (16 sequences)

SEQ ID NO: 1 - DNA, 1943 bp, Homo sapiens
ggaattcccgggtcgacccacgcgtccggtgacggcggtgctgcgaggtcggcgcgcacgtccgccgcgggtcgctcgggcgctgtccaggcggagccggccccgcccgggctgcagccatgatcaagcgtttcctggaggacaccacggatgatggagaactgagcaagttcgtgaaggatttctcaggaaatgcgagctgccacccaccagaggctaagacctgggcatccaggccccaagtcccggagccaaggccccaggccccggacctctatgatgatgacctggagttcagacccccctcgcggccccagtcctctgacaaccagcagtacttctgtgccccagcccctctcagcccatctgccaggccccgcagcccatggggcaagcttgatccctatgattcctctgaggatgacaaggagtatgtgggctttgcaaccctccccaaccaagtccaccgaaagtccgtgaagaaaggctttgactttaccctcatggtggcaggagagtctggcctgggcaaatccacacttgtcaatagcctcttcctcactgatctgtaccgggaccggaaacttcttggtgctgaagagaggatcatgcaaactgtggagatcactaagcatgcagtggacatagaagagaagggtgtgaggctgcggctcaccattgtggacacaccaggttttggggatgcagtcaacaacacagagtgctggaagcctgtggcagaatacattgatcagcagtttgagcagtatttccgagacgagagtggcctgaaccgaaagaacatccaagacaacagggtgcactgctgcctgtacttcatctcacccttcggccatgggtatggtccaagcctgaggctcctggcaccaccgggtgctgtcaagggaacaggccaagagcaccaggggcagggctgccactagcaggtggtcacaggttcctgttccccaggctccggccattggatgttgaattcatgaaggccctgcatcagcgggtcaacatcgtgcctatcctggctaaggcagacacactgacacctcccgaagtggaccacaagaaacgcaaaatccgggaggagattgagcattttggaatcaagatctatcaattcccagactgtgactctgatgaggatgaggacttcaaattgcaggaccaagccctaaaggaaagcatcccatttgcagtaattggcagcaacactgtagtagaggccagagggcggcgagttcggggtcgactctacccctggggcatcgtggaagtggaaaacccagggcactgcgactttgtgaagctgaggacaatgctggtacgtacccacatgcaggacctgaaggatgtgacacgggagacacattatgagaactaccgggcacagtgcatccagagcatgacccgcctggtggtgaaggaacggaatcgcaagtatgaccagaagccaggacaaagctggcagggggagatcccaagcctagccttgggtgagaccaagccctacttttgttcttctataggccctgggctcaatctaagcgggtgctggggtcctcctcgccttatcaacccttttctccctttagcaaactgactcgggaaagtggtaccgacttccccatccctgctgtcccaccagggacagatccagaaactgagaagcttatccgagagaaagatgaggagctgcggcggatgcaggagatgctacacaaaatacaaaaacagatgaaggagaactattaactggctttcagccctggatatttaaatctcctcctcttcttcctgtccatgccggcccctcccagcaccagctctgctcaggccccttcagctactgccacttcgccttacatccctgctgactgcccagagactcagaggaaataaagtttaataaatctgtaggtggctaaaaa

SEQ ID NO: 2 - protein, 274 aa, Homo sapiens
MIKRFLEDTTDDGELSKFVKDFSGNASCHPPEAKTWASRPQVPEPRPQAPDLYDDDLEFRPPSRPQSSDNQQYFCAPAPLSPSARPRSPWGKLDPYDSSEDDKEYVGFATLPNQVHRKSVKKGFDFTLMVAGESGLGKSTLVNSLFLTDLYRDRKLLGAEERIMQTVEITKHAVDIEEKGVRLRLTIVDTPGFGDAVNNTECWKPVAEYIDQQFEQYFRDESGLNRKNIQDNRVHCCLYFISPFGHGYGPSLRLLAPPGAVKGTGQEHQGQGCH

SEQ ID NO: 3 - DNA, 825 bp, Homo sapiens
atgatcaagcgtttcctggaggacaccacggatgatggagaactgagcaagttcgtgaaggatttctcaggaaatgcgagctgccacccaccagaggctaagacctgggcatccaggccccaagtcccggagccaaggccccaggccccggacctctatgatgatgacctggagttcagacccccctcgcggccccagtcctctgacaaccagcagtacttctgtgccccagcccctctcagcccatctgccaggccccgcagcccatggggcaagcttgatccctatgattcctctgaggatgacaaggagtatgtgggctttgcaaccctccccaaccaagtccaccgaaagtccgtgaagaaaggctttgactttaccctcatggtggcaggagagtctggcctgggcaaatccacacttgtcaatagcctcttcctcactgatctgtaccgggaccggaaacttcttggtgctgaagagaggatcatgcaaactgtggagatcactaagcatgcagtggacatagaagagaagggtgtgaggctgcggctcaccattgtggacacaccaggttttggggatgcagtcaacaacacagagtgctggaagcctgtggcagaatacattgatcagcagtttgagcagtatttccgagacgagagtggcctgaaccgaaagaacatccaagacaacagggtgcactgctgcctgtacttcatctcacccttcggccatgggtatggtccaagcctgaggctcctggcaccaccgggtgctgtcaagggaacaggccaagagcaccaggggcagggctgccactag

SEQ ID NO: 4 - protein, 8 aa, Homo sapiens
  MISC_FEATURE (2)..(2): Xaa represents any amino acid
GXSGXGKX

SEQ ID NO: 5 - protein, 26 aa, Homo sapiens
GPSLRLLAPPGAVKGTGQEHQGQGCH

SEQ ID NO: 6 - DNA, 1470 bp, Unknown
  Rat
gcggtggcggccgctctagaatagtggatcctgtatcagatatctcgcatatctgatatttacattatggttcataacgctaccagatttacagttctgaagtagcaacaaaactaattctatggttgggggataccacaacttgtgtaaaaggactgcagcattaggaacatgagaacctctgctctattcagatcaacttaggcctcagccatcaaagtatatgctgtttctaaaggcaagttgctccaaacacctagtctgaaaatagaaacaaacaaacaaaaaatgtgtgtgtaattggccacacactgataccaattgtgcttgtttttgattgaaatggacattgattctctttggggcttgaatcaaaacttttggctttgttcactgacccataattgaaaataattttttcctctggtcgtgaattcttctcccaccccagccttgctccaggattattaaaatcaagtcacatgaaatagcactaatctgtcccggatgaaattttggaattttttttaaatcctcaaacagctcaagtttatctggggctcggaaggaatgaggaactattaataccacaaggttttctttgaaggaagaataagcaggttgtttccttttacatgattagctttcatccctgggcacagcagctggcggtggctgcttgtctttgttttaaacgttctcttccttatttctgttggggaaatgaaaatgttagaaggcaagcattgtggtcatgcccgagcttcaaacatggttctcaggaaatgcgtacagataaaaggcttccttgccttctacacttcagccccttatgagactctaagccaatatggtttgctccttggggaacctgggccctgggagtctttaaattgcctggaaggatttttaaattattttcattttttttctcaaaaagcttcacaggcatcacacatttctgaaaataaaactgccttcattaactagaatgtcatggtatttttgcttttgattagagtcaaattaattcagtttgataacattgattacaagctgatctagagctgtgattagagctttatatatcttagatgattaaatgtggaaataggttaattatgtcttaataaatgcagttgatttgatagaaagaaaattatacaacccgaaattaggcagaaaattatgacaaaatttcagaaattcctgtatacatcttgtacggagtggtaagaccaataaaaatttcaaatgtatctattttagaattcttctggaatcataagattacaaatatttattacttttggtcaccttgatatgaaaaatacagagccaaattatttattctctgtctgcctgtctctctctcaatcacacacacacacacacacacacacacacacacacacacacactgaactaaaccaaaaacacaattatacaaattttatattgaaagaaccc

SEQ ID NO: 7 - DNA, 22 bp, Unknown
  Rat
ggttctcaggaaatgcgtacag

SEQ ID NO: 8 - DNA, 24 bp, Artificial Sequence
  Synthetic
catcttgtacggagtggtaagacc

SEQ ID NO: 9 - DNA, 40 bp, Artificial Sequence
  Synthetic
ggagagtctggcctggagaatcccacacttgtcaatagcc

SEQ ID NO: 10 - protein, 10 aa, Homo sapiens
GRAGSGKSVI

SEQ ID NO: 11 - protein, 10 aa, Homo sapiens
GMAGCGKSVL

SEQ ID NO: 12 - protein, 10 aa, Homo sapiens
GESGLGKSTL

SEQ ID NO: 13 - protein, 10 aa, Homo sapiens
GESGLGKSTL

SEQ ID NO: 14 - protein, 10 aa, Drosophila
GASGLGKSTL

SEQ ID NO: 15 - protein, 10 aa, C. elegans
GRSGLGKSTL

SEQ ID NO: 16 - protein, 10 aa, S. cerevisiae
GQSGLGKSTL

(19) United States
(12) Patent Application Publication
(10) Pub. No.: US 2003/0124571 A1
(43) Pub. Date: Jul. 3, 2003

(54) Novel human septin and uses therefor

(75) Inventors: Sarit Larisch, Zichron-Yaakov (IL); Seong-Jin Kim, Bethesda, MD (US); Robert J. Lechleider, Bethesda, MD (US); Anita B. Roberts, Bethesda, MD (US); Youngsuk Yi, Redwood City, CA (US)

Correspondence Address: MERCHANT & GOULD PC, P.O. BOX 2903, MINNEAPOLIS, MN, 55402-0903, US

(73) Assignee: The Government of the United States of America, Rockville, MD (US)

(21) Appl. No.: 10/208,304

(22) Filed: Jul. 29, 2002

Related U.S. Application Data
(63) Continuation of application No. PCT/US01/02466, filed on Jan. 25, 2001.
(60) Provisional application No. 60/178,866, filed on Jan. 29, 2000.
(60) Provisional application No. 60/258,725, filed on Dec. 29, 2000.

(51) Int. Cl. C12Q 1/68; C07H 21/04; C12N 9/64; C12P 21/02; C12N 5/06

(52) U.S. Cl. 435/6; 435/69.1; 435/226; 435/320.1; 435/325; 536/23.2

(57) ABSTRACT

The invention provides a newly identified and isolated nucleotide sequence encoding a polypeptide referred to in the present application as ARTS, for Apoptosis Related protein in the TGF-β Signaling pathway.

This application is being filed as a PCT International Patent application on Jan. 25, 2001, designating all countries, in the name of The Government of the United States, represented by the Secretary, Department of Health and Human Services (applicant for all countries except the U.S.), and in the names of Sarit Larisch-Bloch, an Israel citizen; Scong-Jin Kim, a U.S. citizen; Robert J. Lechleider, a U.S. citizen; Anita B. Roberts, a U.S. citizen; and Youngsuk Yi, a U.S. citizen (applicants for U.S. only).

This application claims priority to U.S. Provisional Application Serial No. 60/178,866, filed Jan. 29, 2000, entitled NOVEL PRO-APOPTOTIC PROTEIN, ARTS, and to a U.S. Provisional Application Serial No. 60/258,725, filed Dec. 29, 2000, entitled NOVEL HUMAN SEPTIN AND USES THEREFOR, the disclosures of which are hereby incorporated by reference.

### BACKGROUND

TGF-β is a multifunctional protein having a broad spectrum of cellular activities ranging from regulation of target gene activity to control of cell growth and apoptosis (Hsing et al., (1996) Cancer Res. 56, 5146; Rotello et al., (1992) Proc. Natl. Acad. Sci. U.S.A. 88, 3412 (1991); Lin et al., (1992) Cancer Res. 52, 385; Yanagihara et al., Cancer Res. 52, 4042; Sporn et al., (1990) “The multifunctional nature of peptide growth factors” In the Handbook of Experimental Pharmacology, eds. Sporn and Robers, Springer-Verlag, Heidelberg, Vol. 95, pp.3-15). While certain signal transduction pathways of TGF-β are known to be mediated by SMAD proteins (Massagué, (1997) Annu. Rev. Biochem. 67, 753; and Derynck et al., (1998) Biochim. Biophys. Acta 1333, F105) or by MAP kinase pathways (Hartsough et al., (1997) Pharmacol Ther. 75, 21), very little information exists regarding TGF-β apoptotic pathways.

Progressive loss of sensitivity to transforming growth factor-β (TGF-β) growth regulatory signals is a common feature of many different tumor cell types and has been shown to be important in the later stages of disease progression (Wakefield et al., (1995) Prog. Clin. Biol. Res., 391: 133-148; Reiss, M. (1997) Oncol. Res., 9: 447-457; and Markowitz, et al, (1996) Cytokine Growth Factor Rev., 7: 93-102), including human prostatic adenocarcinoma (Williams et al., (1996) Clin. Cancer Res., 2: 635-640). In many cases this loss of sensitivity results from loss of expression or mutational inactivation of the TGF-β receptors (Knaus et al., (1996) Mol. Cell. Biol. 16: 3480-3489; Kim et al., (1996) Cancer Res. 56: 44-48; Choi et al., (1998) J. Biol. Chem. 273: 110-117; and Ammanamanchi et al., (1998) J. Biol. Chem., 273: 16527-16534).

Mutations or allelic loss of molecules involved in TGF-β signaling through the SMAD pathway can also result in resistance to the growth inhibitory effects of TGF-β, even when the receptors are functional (Zhou et al., (1998) Proc. Natl. Acad. Sci. U.S.A., 95: 2412-2416; de Caestecker et al., (1997) J. Biol. Chem., 272: 13690-13696; Moskaluk et al., (1996) Biochim. BioPhys. Acta, 1288: M31-M33; Eppert et al., (1996) Cell, 86: 543-55; Hahn et al., (1996) Science, 271: 350-353; Schutte et al., (1996) Cancer Res., 56: 2527-2530; and Nagatake et al., (1996) Cancer Res., 56: 2718-2720).

### SUMMARY

The invention provides a newly identified and isolated nucleotide sequence encoding a protein product referred to as “ARTS”, for Apoptosis Related protein in the TGF-β Signaling pathway. ARTS is a member of the septin family of proteins and possesses a unique C-terminus and which effectively mediates apoptosis and modulates the actions of TGF-β on cells.

The invention also provides a plasmid or vector that includes the nucleic acid sequence encoding ARTS. The nucleic acid can be operably linked to a promoter that is functional in the vector and provides for expression. The plasmid or vector may also be operably linked to a leader sequence which functions to direct the protein product to a cellular location.

The invention also includes a method of producing ARTS by transfecting a host cell with a plasmid or vector that includes a nucleic acid sequence encoding ARTS.

The invention also provides a method for increasing the sensitivity of a cell to apoptosis induced by TGF-β or a chemotherapeutic agent. Introduction of an amount of ARTS protein or a nucleic acid encoding ARTS into a cell accomplishes this desired result.

Yet another embodiment of the invention is an antisense oligonucleotide that inhibits ARTS protein expression. The antisense oligonucleotide inhibits ARTS expression by hybridizing to ARTS-encoding RNA.

The invention is further directed to methods for reducing the sensitivity of a cell to TGF-β induced apoptosis. The sensitivity of a cell to TGF-β induced apoptosis is reduced by introducing into the cell an amount of an antisense oligonucleotide which effectively inhibits ARTS expression by hybridizing to ARTS-encoding RNA.

The invention also provides a method for detecting the presence of ARTS in a cell and a method for detecting apoptosis of a cell. The method of detecting apoptosis of a cell includes determining the presence of the nucleic acid sequence encoding ARTS or the amino acid sequence of ARTS within the cell, or determining the location of the nucleic acid sequence encoding ARTS or the amino acid sequence of ARTS, within the cell.

Yet another embodiment of the invention is a recombinant protein produced from the nucleic acid sequence encoding ARTS.

The invention is also directed to antibodies which immunoreact with the amino acid sequence of ARTs.

The invention also contemplates a kit for assaying the presence and quantity of ARTS protein or nucleic acid sequence encoding ARTS in a biological or inanimate sample. The kit includes antibodies conjugated to a detectable substrate which immunoreact with the amino acid sequence of ARTS.

The purified compositions of the invention include any compositions containing the nucleic acid sequence encoding ARTS or the amino acid sequence of ARTS obtained from a natural source such as, for example, mammalian sources such as human tissues, including but not limited to human brain and heart tissue, or other mammalian tissues, such as rat prostate epithelial cells. Alternately, the compositions can be synthesized by recombinant or non-recombinant (e.g., automated synthesis) methods.

The above summary of the present invention is not intended to describe each disclosed embodiment or every implementation of the present invention. The Figures and the detailed description that follow more particularly exemplify these embodiments.

### BRIEF DESCRIPTION OF THE FIGURES

FIG. 1A is a bar graph showing the ability of TGF-β to induce the activity of the p8001uc construct.

FIG. 1B is a bar graph showing the ability of TGF-β to induce the activity of the p3TP-Lux construct.

FIG. 2 is a bar graph showing that that activity of transiently transfected cyclin A promoter construct (pCAL2-luc) in NRP-154 and M-NRP1 cells.

FIG. 3 shows thymidine uptake of NRP-154 and M-NRP1 cells after treatment with TGF-β.

FIG. 4 is a schematic showing the alignment of ARTS and hH5/PNUTL2/hcdcrel2a and hcdcrel2b.

FIG. 5 shows the P-loop motif for the apoptogenic proteins CED-4 and Apaf-1 and septin family members (human) ARTS and NEDD5, Drosophila PEANUT, C. elegans C-CDC10 and S. cerevisiae Y-CDC10.

FIG. 6A is a bar graph showing apoptotic response of cells transfected with vector and wt ARTS respond identically.

FIG. 6B is a bar graph showing the caspase 3 activity in NRP-154 cells.

FIG. 7 is a bar graph showing the percent of 200 cells exhibiting nuclear staining of ARTS in COS and NRP-154 cells transfected with either ARTS or M-ARTS.

FIG. 8 shows a bar graph showing apoptosis induced in COS cells when examined using an immunofluorescence assay.

FIG. 9 shows the caspase 3 activity of cells transfected with equal amounts of control or ARTS vector.

FIGS. 10A and 10B show bar graphs demonstrating that ARTS activates the TGF-β responsive 3TP-Lux promoter construct in cells sensitive to TGF-β induced apoptosis.

### DETAILED DESCRIPTION

I. Overview

The present invention provides a newly identified and isolated nucleotide sequence encoding a polypeptide referred to in the present application as ARTS, for Apoptosis Related protein in the TGF-β Signaling pathway. In particular, cDNA encoding an ARTS polypeptide has been identified and isolated, as disclosed in further detail in the Examples below. For sake of simplicity, in the present specification the protein encoded by SEQ. ID. NO: 1, as well as all further native homologues and variants included in the definition of ARTS, provided below, will be referred to as ARTS, regardless of their origin or mode of preparation. The ARTS protein is also described in copending U.S. Provisional Application Serial No. 60/178,866, filed Jan. 29, 2000, the disclosure of which is incorporated herein by reference in its entirety.

The terms “ARTS polypeptide”, “ARTS protein” and “ARTS” when used herein encompass native sequence ARTS and ARTS variants (which are further defined herein). The ARTS protein may be isolated from a variety of sources, such as from human tissue types or from another source, or prepared by recombinant and/or synthetic methods.

The ARTS protein is encoded by an 822 base pair cDNA sequence (SEQ. ID. NO: 1), encoding a predicted polypeptide of 274 amino acids (predicted molecular weight 32 kDa). The experimentally determined molecular weight is 34 kDa, suggesting post-translational modifications of ARTS.

It is believed that ARTS is the product of an alternatively spliced form of the hH5/PNUTL2/hCD-Crel-2 septin family gene in which the 113 bp intervening sequence between exons VI and VII of the H5 gene was not spliced out, generating a new in-frame stop codon. As a result, ARTS shares its N-terminal start site with hCDCrel-2b and has a unique 27 amino acid C-terminus (GPSLRLLAPPGAVKGTGQEHQGQGCH; SEQ. ID. NO: 5) not found in any of the other products of this gene.

The ARTS protein contains a Walker A box P-loop motif. Generally, the consensus sequence of a P-loop motif can be represented by the following: GXSGXGKS/T (SEQ. ID. NO: 4), in which the “X” represent any amino acid, including any of the 20 natural amino acids or a modified amino acid. P-loop motifs are conserved in the septin family of proteins and found in many ATP/GTPases, including the apoptotic regulators CED-4 and Apaf-1. A review of the P-loop motif can be found in Saraste et al., Trends Biochem. Sci., 11:430-434 (1990). Of two other motifs, (G3, DXXG, and G4, XKXD) found in small GTPases and common to most other septins, only the G3 motif is preserved in ARTS, suggesting that it might not bind GTP as shown for other septins. Again, the “X” in these motifs represents any amino acid.

In cells sensitive to TGF-β induced apoptosis, ARTS protein is localized predominantly in the cytoplasm (in particular, the mitochondria). As used herein, the term “sensitive to TGF-β induced apoptosis” means that the cells, when exposed to at least about 1 ng/ml TGF-β, typically between 1 ng/ml to about 10 ng/ml TGF-β, more typically between 1 ng/ml to about 5 ng/ml TGF-β, undergo apoptosis. Apoptosis is cell death typically characterized by condensation and subsequent fragmentation of the cell nucleus. In cells sensitive to TGF-β induced apoptosis, treatment with TGF-β causes ARTS to translocate from the mitochondria to the nucleus.

In contrast, in cells not sensitive to apoptosis induced by TGF-β, ARTS appears to be localized in the nucleus. As used herein, the term “not sensitive to apopotosis induced by TGF-β” means that the cells, when exposed to at least about 1 ng/nl TGF-β (typically between 1 ng/ml and 10 ng/ml TGF-β1 more typically between 1 ng/ml to about 5 ng/ml TGF-β) do not display an apoptotic response. ARTS may also be overexpressed in cells not sensitive to apoptosis induced by TGF-β.

II. Nucleic Acid Sequence

The invention provides an isolated nucleic acid sequence encoding “ARTS” (Apoptosis Related protein in the TGF-β Signaling pathway) shown in Table 1, a sequence complementary to the sequence in Table 1, and a fragment or variant thereof. The 1911 base pair sequence (shown in Table 1) was deposited with the ATCC on Jan. 28, 2000, and has accession number PTA-1235. The sequence was also submitted to GenBank on Jan. 3, 2001, and has accession number BackIt286203 AF176379.

TABLE 1
Nucleic acid and Amino acid sequence for ARTS

| | | | |
|---|---|---|---|
| | atgatcaagcgtttcctggaggacaccacggatgatggagaactg | | (SEQ. ID. NO:1) |
| | M I K R F L E D T T D D G E L | (SEQ. ID. NO:2) | |
| 133 | agcaagttcgtgaaggatttctcaggaaatgcgagctgccaccca | | |
| | S K F V K D F S G N A S C H P | | |
| 178 | ccagaggctaagacctgggcatccaggccccaagtcccggagcca | | |
| | P E A K T W A S R P Q V P E P | | |
| 223 | aggccccaggccccggacctctatgatgatgacctggagttcaga | | |
| | R P Q A P D L Y D D D L E F R | | |
| 268 | cccccctcgcggccccagtcctctgacaaccagcagtacttctgt | | |
| | P P S R P Q S S D N Q Q Y F C | | |
| 313 | gccccagcccctctcagcccatctgccaggccccgcagcccatgg | | |
| | A P A P L S P S A R P R S P W | | |
| 358 | ggcaagcttgatccctatgattcctctgaggatgacaaggagtat | | |
| | G K L D P Y D S S E D D K E Y | | |
| 403 | gtgggctttgcaaccctccccaaccaagtccaccgaaagtccgtg | | |
| | V G F A T L P N Q V H R K S V | | |
| 448 | aagaaaggctttgactttaccctcatggtggcaggagagtctggc | | |
| | K K G F D F T L M V A G E S G | | |
| 493 | ctgggcaaatccacacttgtcaatagcctcttcctcactgatctg | | |
| | L G K S T L V N S L F L T D L | | |
| 538 | taccgggaccggaaacttcttggtgctgaagagaggatcatgcaa | | |
| | Y R D R K L L G A E E R I M Q | | |
| 583 | actgtggagatcactaagcatgcagtggacatagaagaqaagggt | | |
| | T V E I T K H A V D I E E K G | | |
| 628 | gtgaggctgcggctcaccattgtggacacaccaggttttggggat | | |
| | V R L R L T I V D T P G F G D | | |
| 673 | gcagtcaacaacacagagtgctggaagcctgtggcagaatacatt | | |
| | A V N N T E C W K P V A E Y I | | |
| 718 | gatcagcagtttgagcagtatttccgagacgagagtggcctgaac | | |
| | D Q Q F E Q Y F R D E S G L N | | |
| 763 | cgaaagaacatccaagacaacagggtgcactgctgcctgtacttc | | |
| | R K N I Q D N R V H C C L Y F | | |
| 808 | atctcacccttcggccatgggtatggtccaagcctgaggctcctg | | |
| | I S P F G H G Y G P S L R L L | | |
| 853 | gcaccaccgggtgctgtcaagggaacaggccaagagcaccagggg | | |
| | A P P G A V K G T G Q E H Q G | | |
| 898 | cagggctgccactag | 912 | |
| | Q G C H * | | |

The term “oligonucleotide” or “nucleic acid sequence” refers to an oligomer or polymer of ribonucleic acid (RNA) or deoxyribonucleic acid (DNA) or mimetics thereof. This term includes oligonucleotides composed of naturally-occurring nucleobases, sugars and covalent internucleoside (backbone) linkages as well as oligonucleotides having non-naturally-occurring portions which function similarly. Such modified or substituted oligonucleotides are often preferred over native forms because of desirable properties such as, for example, enhanced cellular uptake, enhanced affinity for nucleic acid target and increased stability in the presence of nucleases.

As used herein, the term “isolated nucleic acid sequence” refers to a nucleic acid, including both DNA and/or RNA, which in some way is not identical to that of any naturally occurring nucleic acid or to that of any naturally occurring genomic nucleic acid. The term therefore covers, for example, (a) DNA that has the sequence of part of a naturally occurring genomic DNA molecule, but is not flanked by both of the coding sequences that flank the DNA in the genome of the organism in which it naturally occurs; (b) a nucleic acid incorporated into a vector or into the genomic DNA of a prokaryote or eukaryote in a manner such that that resulting molecule is not identical to any naturally occurring vector or genomic DNA; (c) a separate molecule such as a cDNA, a genomic fragment, a fragment (either DNA or RNA) produced by polymerase chain reaction (PCR), or a restriction fragment; and (d) a recombinant nucleic acid sequence that is part of a hybrid gene (i.e., a gene encoding a fusion protein). The term “isolated” may also be used interchangeably with the term “purified.”

As used herein, the terms “complementary” or “complement”, when used in reference to a nucleic acid sequence, refers to sequences that are related by the base-pairing rules developed by Watson and Crick. For example, for the sequence “T-G-A” the complementary sequence is “A-C-T.”

The invention also includes nucleic acid sequences that are capable of hybridizing to to all or a portion of the nucleic acid sequence represented by SEQ. ID. NO. 1, or its complement, under stringent or moderately stringent hybridization conditions (as defined herein). The term “hybridizing” refers to the pairing of complementary nucleic acids. Hybridization” can include hydrogen bonding, which may be Watson-Crick, Hoogsteen or reversed Hoogsteen hydrogen bonding, between complementary nucleoside or nucleotide bases. Hybridization and the strength of hybridization (i.e., the strength of the association between the nucleic acids) is influenced by such factors as the degree of complementarity between the nucleic acids, stringency of the conditions involved, the melting temperature (Tₘ) of the formed hybrid, and the G:C ratio within the nucleic acids. Complementarity may be “partial,” in which only some of the bases of the nucleic acids are matched according to the base pairing rules. Alternatively, there may be “complete” or “total” complementarity between the nucleic acids. The degree of complementarity between the nucleic acid strands has effects on the efficiency and strength of hybridization between the nucleic acid strands. If desired, the hybridizing sequence can include a label, such as a radiolabel (e.g., ³H, ¹⁴C, ³²P or ¹²⁵I, etc.) or a fluorescent label (e.g., fluorescein, rhodamine, etc.).

The hybridizing portion of the hybridizing nucleic acids is typically at least 15 (e.g., 20, 25, 30 or 50) nucleotides in length and at least 80% (e.g., at least 95% or at least 98%) identical to the sequence of SEQ. ID. NO: 1, or its complement. Hybridizing nucleic acids of the type described herein can be used, for example, as a cloning probe, a primer (e.g., a PCR primer), or a diagnostic probe. Hybridization of the oligonucleotide probe to a nucleic acid sample typically is performed under stringent conditions.

Nucleic acid duplex or hybrid stability is expressed as the melting temperature or Tm, which is the temperature at which a probe dissociates from a target DNA. This melting temperature is used to define the required stringency conditions. If sequences are to be identified that are related and substantially identical to the probe, rather than identical, then it is useful to first establish the lowest temperature at which only homologous hybridization occurs with a particular concentrion of salt (e.g., SSC or SSPE). Then, assuming that 1% mismatching results in a 1° C. decrease in the Tm, the temperature of the final wash in the hybridization reaction is reduced accordingly (for example, if sequences having >95% identity with the probe are sought, the final wash temperature is decreased by 5° C. In practice, the change in Tm can be between 0.5° C. and 1.5° C. per 1% mismatch. As used herein, “stringent conditions” involve hybridizing at 68° C. in 5× SSC/5× Denhardt's solution/1.0% SDS, and washing in 0.2× SSC/0.1% SDS at room temperature. “Moderately stringent” conditions include washing in 3× SSC at 42° C. The parameters of salt concentration and temperature can be varied to achieve the optimal level of identity between the probe and the target nucleic acid. Additional guidance regarding such conditions is readily available, for example, by Sambrook et al, 1989, Molecular Clonging, A Laboratory Manual, Cold Spring Harbor Press, N.Y.

As used herein, the term “percent homology” or “percent identity” of two nucleic acid sequences is determined using the algorithm of Karlin and Altschul (1990) Proc. Natl. Acad. Sci. USA 87: 2264-2268, modified as in Karlin and Altschul (1993) Proc. Natl. Acad. Sci. USA 90: 5873-5877. Such an algorithm is incorporated into the NBLAST program of Altschul et al. (1990) J. Mol. BiOl. 215: 402-410. To obtain gapped alignments for comparision purposes, Gapped BLAST is used as described by Altschul et al. (1997) Nuelic Acids Res. 25: 3389-3402. When using BLAST and gapped BLAST programs, the default parameters of the respective programs (e.g., NBLAST) are used.

The invention also includes degenerate variants of the nucleic acid sequence shown in SEQ. ID. NO: 1. The genetic code is made up of sixty-four codons. Three code for chain termination. The remaining sixty-one triplets encode the twenty amino acids. Many amino acids are coded by more than one codon. Thus, the genetic code is said to be degenerate. A “degenerate variant” refers to a nucleic acid sequence in which a codon in the nucleic acid sequence, which codes for a particular amino acid, is exchanged for another codon that codes for the same amino acid. For example, in a degenerate variant, the sequence ACU, coding for threonine, may be exchanged for the sequence ACC, which also codes for threonine. See, for example, Stryer, (1988) Biochemistry, W. H. Freeman and Co., New York, Chapter 5, page 107, Table 5.5. If desired, one or more such exchanges can be made in a degenerate variant.

As is known in the art, a nucleoside is a base-sugar combination. The base portion of the nucleoside is normally a heterocyclic base. The two most common classes of such heterocyclic bases are the purines and the pyrimidines. Nucleotides are nucleosides that further include a phosphate group covalently linked to the sugar portion of the nucleoside. For those nucleosides that include a pentofuranosyl sugar, the phosphate group can be linked to either the 2′, 3′ or 5′ hydroxyl moiety of the sugar. In forming oligonucleotides, the phosphate groups covalently link adjacent nucleosides to one another to form a linear polymeric compound. In turn the respective ends of this linear polymeric structure can be further joined to form a circular structure, however, open linear structures are generally preferred. Within the oligonucleotide structure, the phosphate groups are commonly referred to as forming the internucleoside backbone of the oligonucleotide. The normal linkage or backbone of RNA and DNA is a 3′ to 5′ phosphodiester linkage.

The invention also includes oligonucleotides containing modified backbones or non-natural internucleoside linkages. As defined in this specification, oligonucleotides having modified backbones include those that retain a phosphorus atom in the backbone and those that do not have a phosphorus atom in the backbone. For the purposes of this specification, and as sometimes referenced in the art, modified oligonucleotides that do not have a phosphorus atom in their internucleoside backbone can also be considered to be oligonucleosides.

Preferred modified oligonucleotide backbones include, for example, phosphorothioates, chiral phosphorothioates, phosphorodithioates, phosphotriesters, aminoalkylphosphotriesters, methyl and other alkyl phosphonates including 3′-alkylene phosphonates and chiral phosphonates, phosphinates, phosphoramidates including 3′-amino phosphoramidate and aminoalkylphosphoramidates, thionophosphoramidates, thionoalkylphosphonates, thionoalkylphosphotriesters, and boranophosphates having normal 3′-5′ linkages, 2′-5′ linked analogs of these, and those having inverted polarity wherein the adjacent pairs of nucleoside units are linked 3′-5′ to 5′-3′ or 2′-5′ to 5′-2′. Various salts, mixed salts and free acid forms are also included.

Preferred modified oligonucleotide backbones that do not include a phosphorus atom therein have backbones that are formed by short chain alkyl or cycloalkyl internucleoside linkages, mixed heteroatom and alkyl or cycloalkyl internucleoside linkages, or one or more short chain heteroatomic or heterocyclic internucleoside linkages. These include those having morpholino linkages (formed in part from the sugar portion of a nucleoside); siloxane backbones; sulfide, sulfoxide and sulfone backbones; formacetyl and thioformacetyl backbones; methylene formacetyl and thioformacetyl backbones; alkene containing backbones; sulfamate backbones; methyleneimino and methylenehydrazino backbones; sulfonate and sulfonamide backbones; amide backbones; and others having mixed N, O, S and CH.sub.2 component parts.

In other preferred oligonucleotide mimetics, both the sugar and the internucleoside linkage, i.e., the backbone, of the nucleotide units are replaced with novel groups. The base units are maintained for hybridization with an appropriate nucleic acid target compound. One such oligomeric compound, an oligonucleotide mimetic that has been shown to have excellent hybridization properties, is referred to as a peptide nucleic acid (PNA). In PNA compounds, the sugar-backbone of an oligonucleotide is replaced with an amide containing backbone, in particular an aminoethylglycine backbone. The nucleobases are retained and are bound directly or indirectly to aza nitrogen atoms of the amide portion of the backbone.

The invention also includes oligonucleotides with phosphorothioate backbones and oligonucleosides with heteroatom backbones. Modified oligonucleotides may also contain one or more substituted sugar moieties.

The oligonucleotides of the invention may also include nucleobase (often referred to in the art simply as “base”) modifications or substitutions. As used herein, “unmodified” or “natural” nucleobases include the purine bases adenine (A) and guanine (G), and the pyrimidine bases thymine (T), cytosine (C) and uracil (U). Modified nucleobases include other synthetic and natural nucleobases such as 5-methylcytosine (5-me-C), 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl uracil and cytosine, 6-azo uracil, cytosine and thymine, 5-uracil (pseudouracil), 4-thiouracil, 8-halo, 8-amino, 8-thiol, 8-thioalkyl, 8-hydroxyl and other 8-substituted adenines and guanines, 5-halo particularly 5-bromo, 5-trifluoromethyl and other 5-substituted uracils and cytosines, 7-methylguanine and 7-methyladenine, 8-azaguanine and 8-azaadenine, 7-deazaguanine and 7-deazaadenine and 3′-deazaguanine and 3′-deazaadenine.

Another modification of the oligonucleotides of the invention involves chemically linking to the oligonucleotide one or more moieties or conjugates to enhance the activity, cellular distribution or cellular uptake of the oligonucleotide.

It is not necessary for all positions in a given compound to be uniformly modified, and in fact more than one of the aforementioned modifications may be incorporated in a single compound or even at a single nucleoside within an oligonucleotide.

DNA encoding ARTS protein may be obtained from a cDNA library prepared from tissue believed to possess the ARTS mRNA and to express it at a detectable level. Accordingly, human DNA can be conveniently obtained from a cDNA library prepared from human tissue. The ARTS-encoding gene may also be obtained from a genomic library or by oligonucleotide synthesis.

Libraries can be screened with probes (such as antibodies to the ARTS protein or oligonucleotides of at least about 20-80 bases) designed to identify the gene of interest or the protein encoded by it. Screening the cDNA or genomic library with the selected probe may be conducted using standard procedures, such as described in Sambrook et al., Molecular Cloning: A Laboratory Manual (New York: Cold Spring Harbor Laboratory Press, 1989). An alternative means to isolate the gene encoding ARTS is to use PCR methodology (Sambrook et al., supra; Dieffenbach et al., PCR Primer: A Laboratory Manual, Cold Spring Harbor Laboratory Press, 1995).

When screening a cDNA library, the oligonucleotide sequences selected as probes should be of sufficient length and sufficiently unambiguous that false positives are minimized. The oligonucleotide is preferably labeled such that it can be detected upon hybridization to DNA in the library being screened. Methods of labeling are well known in the art, and include the use of radiolabels such as ³²P-labeled ATP, biotinylation or enzyme labeling. Hybridization conditions, including moderate stringency and high stringency, are provided in Sambrook et al., supra, and are described herein.

Nucleotide sequences (or their complement) encoding ARTS have various applications in the art of molecular biology, including uses as hybridization probes, in chromosome and gene mapping and in the generation of anti-sense RNA and DNA. ARTS nucleic acid will also be useful for the preparation of ARTS polypeptides by the recombinant techniques described herein.

The full-length native sequence ARTS gene (SEQ. ID. NO: 1), or fragments thereof, may be used as, among other things, hybridization probes for a cDNA library to isolate other genes (for instance, those encoding naturally-occurring variants of ARTS or ARTS from other species) which have a desired sequence identity to the ARTS sequence disclosed in Table 1 (SEQ. ID. NO: 1). Typically, the length of the probes will be about 20 to about 80 bases. The hybridization probes may be derived from the nucleotide sequence of SEQ. ID. NO: 1 or from genomic sequences including promoters, enhancer elements and introns of native sequence ARTS. By way of example, a screening method may comprise isolating the coding region of the ARTS gene using the known DNA sequence to synthesize a selected probe of about 40 bases. Hybridization probes may be labeled by a variety of labels, including radionucleotides such as ³²P or ³⁵S, or enzymatic labels such as alkaline phosphatase coupled to the probe via avidin/biotin coupling systems. Labeled probes having a sequence complementary to that of the ARTS gene of the present invention can be used to screen libraries of human cDNA, genomic DNA or mRNA to determine which members of such libraries the probe hybridizes to.

Fragments of ARTS DNA contemplated by the invention include sequences comprising at least about 20 to 30 consecutive nucleotides of the DNA of SEQ. ID NO: 1. Preferably, such sequences comprise at least about 50 consecutive nucleotides of the DNA of SEQ. ID. NO: 2.

The probes may also be employed in PCR techniques to generate a pool of sequences for identification of closely related ARTS coding sequences.

Nucleotide sequences encoding ARTS can also be used to construct hybridization probes for mapping the gene that encodes ARTS. The nucleotide sequences provided herein may be mapped to a chromosome and specific regions of a chromosome using known techniques, such as in situ hybridization, linkage analysis against known chromosomal markers, and hybridization screening with libraries.

III. Vectors

The invention also includes expression vectors containing the nucleic acid sequence of SEQ. ID. NO: 1, its complement, variants and/or fragments thereof. As used herein, the term “expression vector” refers to a construct containing a nucleic acid sequence that is operably linked to a suitable control sequence capable of effecting expression of the nucleic acid sequence in a suitable host.

Nucleic acid is “operably linked” when it is placed into a functional relationship with another nucleic acid sequence. For example, DNA sequence encoding a promoter or enhancer is operably linked to a coding sequence if it affects the transcription of the sequence; or a ribosome binding site is operably linked to a coding sequence if it is positioned so as to facilitate translation. Generally, “operably linked” means that the DNA sequences being linked are contiguous, and, in some cases, contiguous and in reading phase. However, some sequences, such as enhancers, do not have to be contiguous. Linking is accomplished by ligation at convenient restriction sites. If such sites do not exist, the synthetic oligonucleotide adaptors or linkers are used in accordance with conventional practice.

The term “control sequences” refers to DNA sequences necessary for the expression of an operably linked coding sequence in a particular host organism. The control sequences that are suitable for prokaryotes, for example, include a promoter, optionally an operator sequence, and a ribosome binding site. Eukaryotic cells are known to utilize promoters, polyadenylation signals, and enhancers.

The nucleic acid (e.g., cDNA or genomic DNA) encoding ARTS may be inserted into a replicable vector for cloning (amplification of the DNA) or for expression. As used herein, “expression vector” means a DNA construct including a DNA sequence (e.g., a sequence encoding a fluorescent protein) that is operably linked to a suitable control sequence (e.g. all or part of a mutagen sensitive gene) capable of affecting the expression of the DNA in a suitable host. Such control sequences may include a promoter to affect transcription, an optional operator sequence to control transcription, a sequence encoding suitable ribosome-binding sites on the mRNA, and sequences that control termination of transcription and translation. Different cell types may be employed with different expression vectors. The vector may be a plasmid, a phage particle, or simply a potential genomic insert. Once transformed into a suitable host, the vector may replicate and function independently of the host genome, or may, under suitable conditions, integrate into the genome itself. In the present specification, plasmid and vector are sometimes used interchangeably. However, the invention is intended to include other forms of expression vectors that serve equivalent functions and which are, or become, known in the art. Useful expression vectors, for example, can include segments of chromosomal, non-chromosomal and synthetic DNA sequences such as various known derivatives of known bacterial plasmids, e.g., plasmids from E. coli including Col E1, pCR1, pBR322, pMb9, pUC 19 and their derivatives, wider host range plasmids, e.g., RP4, phage DNAs e.g., the numerous derivatives of phage 11, e.g., NM989, and other DNA phages, e.g., M13 and filamentous single stranded DNA phages, yeast plasmids such as the 2 mm plasmid or derivatives thereof, vectors useful in eukaryotic cells, such as vectors useful in animal cells and vectors derived from combinations of plasmids and phage DNAs, such as plasmids which have been modified to employ phage DNA or other expression control sequences. Other suitable vectors include viral vectors based on Adeno Associated Virus (AAV) serotypes and viral vectors with adenovirus, retrovirus, and as chimeric virus backbones, e.g., adeno-retroviral or retro-adenoviral vectors. In constructing vectors based on Adeno Associated Virus serotypes, the vectors may be derived from publicly available nucleic acid sequences: AAV2 (GenBank AFo43303 and J01901); AAV4 (WO 98/11244, published Mar. 19, 1998); AAV5 (WO 99/61601, published Dec. 2, 1999).

Expression techniques using the expression vectors of the present invention are known in the art and are described generally in, for example, Sambrook et al., Molecular Cloning: A Laboratory Manual, Second Edition, Cold Spring Harbor Press (1989).

IV. Antisense DNA

The present invention also provides oligomeric antisense compounds, particularly oligonucleotides, for use in modulating the function of nucleic acid molecules encoding ARTS, ultimately modulating the amount of ARTS produced. This is accomplished by providing antisense compounds that specifically hybridize with one or more nucleic acids encoding ARTS. As used herein, the terms “target nucleic acid” and “nucleic acid encoding ARTS” encompass DNA encoding ARTS, RNA (including pre-mRNA and mRNA) transcribed from such DNA, and also cDNA derived from such RNA. The specific hybridization of an oligomeric compound with its target nucleic acid interferes with the normal function of the nucleic acid. This modulation of function of a target nucleic acid by compounds that specifically hybridize to it is generally referred to as “antisense.” More specifically, the invention includes “antisense” DNA or RNA corresponding to the sequence in SEQ. ID. NO: 1, its complement, variants and/or fragments thereof.

The functions of DNA to be interfered with include replication and transcription. The functions of RNA to be interfered with include all vital functions such as, for example, translocation of the RNA to the site of protein translation, translation of protein from the RNA, splicing of the RNA to yield one or more mRNA species, and catalytic activity which may be engaged in or facilitated by the RNA. The overall effect of such interference with target nucleic acid function is modulation of the expression of ARTS. In the context of the present invention, “modulation” means either an increase (stimulation) or a decrease (inhibition) in the expression of a gene.

It is preferred to target specific nucleic acids for antisense. “Targeting” an antisense compound to a particular nucleic acid, in the context of this invention, is a multistep process. The process usually begins with the identification of a nucleic acid sequence whose function is to be modulated. This may be, for example, a cellular gene (or mRNA transcribed from the gene) whose expression is associated with a particular disorder or disease state, or a nucleic acid molecule from an infectious agent. In the present invention, the target is a nucleic acid molecule encoding ARTS. The targeting process also includes determination of a site or sites within this gene for the antisense interaction to occur such that the desired effect, e.g., detection or modulation of expression of the protein, will result.

One target site is the region encompassing the translation initiation or termination codon of the open reading frame (ORF) of the gene. Since, as is known in the art, the translation initiation codon is typically 5′-AUG (in transcribed mRNA molecules; 5′-ATG in the corresponding DNA molecule), the translation initiation codon is also referred to as the “AUG codon,” the “start codon” or the “AUG start codon.” A minority of genes have a translation initiation codon having the RNA sequence 5′-GUG, 5′-UUG or 5′-CUG, and 5′-AUA, 5′-ACG and 5′-CUG have been shown to function in vivo. Thus, the terms “translation initiation codon” and “start codon” can encompass many codon sequences, even though the initiator amino acid in each instance is typically methionine (in eukaryotes) or formylmethionine (in prokaryotes). It is also known that eukaryotic and prokaryotic genes may have two or more alternative start codons, any one of which may be preferentially utilized for translation initiation in a particular cell type or tissue, or under a particular set of conditions. In the context of the invention, “start codon” and “translation initiation codon” refer to the codon or codons that are used in vivo to initiate translation of an mRNA molecule transcribed from a gene encoding ARTS, regardless of the sequence(s) of such codons.

Another possible target site includes the region encompassing a translation termination codon (or “stop codon”) of a gene, having one of three sequences, i.e., 5′-UAA, 5′-UAG and 5′-UGA (the corresponding DNA sequences are 5′-TAA, 5′-TAG and 5′-TGA, respectively). The terms “start codon region” and “translation initiation codon region” refer to a portion of such an mRNA or gene that encompasses from about 25 to about 50 contiguous nucleotides in either direction (i.e., 5′ or 3′) from a translation initiation codon. Similarly, the terms “stop codon region” and “translation termination codon region” refer to a portion of such an mRNA or gene that encompasses from about 25 to about 50 contiguous nucleotides in either direction (i.e., 5′ or 3′) from a translation termination codon.

The open reading frame (ORF) or “coding region,” refers to the region between the translation initiation codon and the translation termination codon, is also a region which may be targeted effectively. Other target regions include the 5′ untranslated region (5′UTR), known in the art to refer to the portion of an mRNA in the 5′ direction from the translation initiation codon, and thus including nucleotides between the 5′ cap site and the translation initiation codon of an mRNA or corresponding nucleotides on the gene, and the 3′ untranslated region (3′UTR), known in the art to refer to the portion of an mRNA in the 3′ direction from the translation termination codon, and thus including nucleotides between the translation termination codon and 3′ end of an mRNA or corresponding nucleotides on the gene. The 5′ cap of an mRNA comprises an N7-methylated guanosine residue joined to the 5′-most residue of the mRNA via a 5′-5′ triphosphate linkage. The 5′ cap region of an mRNA is considered to include the 5′ cap structure itself as well as the first 50 nucleotides adjacent to the cap. The 5′ cap region may also be a preferred target region.

Although some eukaryotic mRNA transcripts are directly translated, many contain one or more regions, known as “introns,” which are excised from a transcript before it is translated. The remaining (and therefore translated) regions are known as “exons” and are spliced together to form a continuous mRNA sequence. mRNA splice sites, i.e., intron-exon junctions, may also be target regions, and are particularly useful in situations where aberrant splicing is implicated in disease, or where an overproduction of a particular mRNA splice product is implicated in disease. Aberrant fusion junctions due to rearrangements or deletions are also preferred targets. It has also been found that introns can also be effective, and therefore preferred, target regions for antisense compounds targeted, for example, to DNA or pre-mRNA.

Once one or more target sites have been identified, oligonucleotides are chosen which hybridize to the target site under stringent or moderately stringent conditions.

Generally, antisense compounds in accordance with the invention preferably comprise from about 8 to about 30 nucleobases (i.e. from about 8 to about 30 linked nucleosides). Particularly preferred antisense compounds are antisense oligonucleotides comprising from about 12 to about 25 nucleobases.

The antisense compounds used in accordance with this invention may be conveniently and routinely made through the well-known technique of solid phase synthesis. Equipment for such synthesis is sold by several vendors including, for example, Applied Biosystems (Foster City, Calif.). Any other means for such synthesis known in the art may additionally or alternatively be employed.

The antisense compounds may also be admixed, encapsulated, conjugated or otherwise associated with other molecules, molecule structures or mixtures of compounds, as for example, liposomes, receptor targeted molecules, oral, rectal, topical or other formulations, for assisting in uptake, distribution and/or absorption.

The antisense compounds can be used for diagnostics, therapeutics, prophylaxis and as research reagents and kits. Antisense compounds are commonly used as research reagents and diagnostics. For example, antisense oligonucleotides that are able to inhibit gene expression with specificity may be used to elucidate the function of a particular gene. Antisense compounds can also be used, for example, to distinguish between functions of various members of a biological pathway. Antisense modulation has, therefore, been harnessed for research use.

Antisense technology can also be used for therapeutic treatment of disease in mammals, including humans. For example a mammal suspected of having a disease or disorder that can be treated by modulating the expression of ARTS can be treated by administering an antisense compound of the invention. The compounds of the invention can be used in pharmaceutical compositions by adding an effective amount of an antisense compound to a suitable pharmaceutically acceptable diluent or carrier. Use of the antisense compounds and methods of the invention may also be useful prophylactically, e.g., to prevent or delay infection, inflammation or tumor formation, for example.

The antisense compounds of the invention are also useful for research and diagnostics, because these compounds hybridize to nucleic acids encoding ARTS, enabling sandwich and other assays to easily be constructed to exploit this fact. Hybridization of the antisense oligonucleotides of the invention with a nucleic acid encoding ARTS can be detected by means known in the art. Such means may include conjugation of an enzyme to the oligonucleotide, radiolabelling of the oligonucleotide or any other suitable detection means. Kits using such detection means for detecting the level of ARTS in a sample may also be prepared.

The present invention also includes pharmaceutical compositions and formulations that include the antisense compounds of the invention. The pharmaceutical compositions of the present invention may be administered in a number of ways depending upon whether local or systemic treatment is desired and upon the area to be treated. Administration may be topical (including ophthalmic and to mucous membranes including vaginal and rectal delivery), pulmonary, e.g., by inhalation or insufflation of powders or aerosols, including by nebulizer; intratracheal, intranasal, epidermal and transdermal), oral or parenteral. Parenteral administration includes intravenous, intraarterial, subcutaneous, intraperitoneal or intramuscular injection or infusion; or intracranial, e.g., intrathecal or intraventricular, administration. Oligonucleotides with at least one 2′-O-methoxyethyl modification are believed to be particularly useful for oral administration.

Delivery of antisense oligonucleotide may also be accomplished by introducing into a cell a DNA expression vector that is transcribed into multiple copies of antisense RNA. Antisense oligonucleotides provide a precise means to shut down expression of a specific gene.

In another embodiment, compositions of the invention may contain one or more antisense compounds, targeted to a first nucleic acid sequence and one or more additional antisense compounds targeted to a second nucleic acid target sequence. Two or more combined compounds may be used together or sequentially. Various publications describe current antisense technology, including WO 00/00504 (Jan. 6, 2000); WO 99/60166 (Nov. 25, 1999); WO 99/50409 (Oct. 7, 1999); and WO 97/25444 (Sep. 17, 1997).

Thus, antisense oligonucleotides can be used to decrease the sensitivity of a cell to TGF-β induced apoptosis. Thus antisense oligonucleotides could be used to reduce the symptoms of clinical syndromes characterized by excessive apoptosis, for example, age-related macular degeneration, and acute quadriplegic myopahty.

V. Amino Acid Sequence

The invention also provides an isolated ARTS protein having the amino acid sequence shown in Table 2, and fragments or variants thereof.

TABLE 2

Amino Acid Sequence of the ARTS Protein

(SEQ. ID. NO:2)

| | |
|---|---|
| 1 | MIKRFLEDTTDDGELSKFVKDFSGNASCHPPEAKTWAS |
| 39 | RPQVPEPRPQAPDLYDDDLEFRPPSRPQSSDNQQYFCA |
| 77 | PAPLSPSARPRSPWGKLDPYDSSEDDKEYVGFATLPNQ |
| 115 | VHRKSVKKGFDFTLMVAGESGLGKSTLVNSLFLTDLYR |
| 153 | DRLLGAEERIMQTVEITKHAVDIEEKGVRLRLTIVDT |
| 191 | PGFGDAVNNTECWKPVAEYIDQQFEQYFRDESGLNRKN |
| 229 | IQDNRVHCCLYFISPFGHGYGPSLRLLAPPGAVGTGQ |
| 267 | EHQGQGCH |

“Isolated,” when used to describe the various polypeptides disclosed herein, means polypeptide that has been identified and separated and/or recovered from a component of its natural environment. Contaminant components of its natural environment are materials that would typically interfere with diagnostic or therapeutic uses for the polypeptide, and may include enzymes, hormones, and other proteinaceous or non-proteinaceous solutes. In preferred embodiments, the polypeptide will be purified (1) to a degree sufficient to obtain at least 15 residues of N-terminal or internal amino acid sequence by use of a spinning cup sequenator, or (2) to homogeneity by SDS-PAGE under non-reducing or reducing conditions using Coomassie blue or, preferably, silver stain. Isolated polypeptide includes polypeptide in situ within recombinant cells, since at least one component of the ARTS natural environment will not be present. Ordinarily, however, isolated polypeptide will be prepared by at least one purification step.

A “native sequence ARTS” comprises a polypeptide having the same amino acid sequence as ARTS derived from nature. Such native sequence ARTS can be isolated from nature or can be produced by recombinant and/or synthetic means. The term “native sequence ARTS” specifically encompasses naturally-occurring truncated forms, naturally-occurring variant forms (e.g., alternatively spliced forms) and naturally-occurring allelic variants of ARTS. In one embodiment of the invention, the native sequence ARTS is a mature or full-length native sequence ARTS comprising amino acids 1 to 274 of Table 2 (SEQ. ID. NO: 2).

A. Variants

In addition to the full-length native sequence ARTS polypeptides described herein, it is contemplated that ARTS variants can be prepared. “ARTS variant” means anything other than a native sequence ARTS, and includes ARTS having at least about 80% amino acid sequence identity with the amino acid sequence of residues 1 to 274 of the ARTS polypeptide having the deduced amino acid sequence shown in Table 2 (SEQ. ID. NO: 2). Such ARTS variants include, for instance, ARTS polypeptides wherein one or more amino acid residues are added, or deleted, at the N- or C-terminus, as well as within one or more internal domains, of the sequence of Table 2 (SEQ. ID. NO: 2). Ordinarily, an ARTS variant will have at least about 80% amino acid sequence identity, more preferably at least about 85% amino acid sequence identity, even more preferably at least about 90% amino acid sequence identity, and most preferably at least about 95% sequence identity with the amino acid sequence of residues 1 to 274 of Table 2 (SEQ. ID. NO: 2). Preferred variants retain the activity of wild type ARTS.

ARTS variants include variants containing conservative amino acid substitutions. As used herein, “conservative amino acid substitution” refers to a replacement of one or more amino acid residue with a different residue having a sidechain with at least one similar biochemical characteristic, such as size, shape, charge or polarity.

“Active” or “activity” for the purposes herein refers to form(s) of ARTS that retain the biologic and/or immunologic activities of native or naturally-occurring ARTS. A preferred activity is the ability to respond to treatment with TGF-β by translocating to the nucleus and causing apoptosis.

As used herein, the term “percent homology” or “percent identity” of two amino acid sequences is determined using the algorithm of Karlin and Altschul (1990) Proc. Natl. Acad. Sci. USA 87: 2264-2268, modified as in Karlin and Altschul (1993) Proc. Natl. Acad. Sci. USA 90: 5873-5877. Such an algorithm is incorporated into the XBLAST programs of Altschul et al. (1990) J. Mol. BiOL. 215: 402-410. BLAST protein searches are performed with the XBLAST program to obtain amino acid sequences homologous to a reference polypeptide. To obtain gapped alignments for comparision purposes, Gapped BLAST is used as described by Altschul et al. (1997) Nuelic Acids Res. 25: 3389-3402. When using BLAST and gapped BLAST programs, the default parameters of the respective programs (e.g., XBLAST) are used.

ARTS variants can be prepared by introducing appropriate nucleotide changes into the ARTS DNA, and/or by synthesis of the desired ARTS polypeptide. Those skilled in the art will appreciate that amino acid changes may alter post-translational processes of the ARTS, such as changing the number or position of glycosylation sites or altering the membrane anchoring characteristics.

Variations in the native full-length sequence ARTS or in various domains of the ARTS described herein, can be made, for example, using known techniques for conservative and non-conservative mutations. Variations may be a substitution, deletion or insertion of one or more codons encoding the ARTS protein that results in a change in the amino acid sequence of ARTS as compared with the native sequence for ARTS. Optionally the variation is by substitution of at least one amino acid with any other amino acid in one or more of the domains of the ARTS protein. Guidance in determining which amino acid residue may be inserted, substituted or deleted without adversely affecting the desired activity may be found by comparing the sequence of the ARTS with that of homologous known protein molecules and minimizing the number of amino acid sequence changes made in regions of high homology. For example, it may be preferably to minimize the number of amino acid sequence changes made in the P-loop domain. Amino acid substitutions can be the result of replacing one amino acid with another amino acid having similar structural and/or chemical properties, such as the replacement of a leucine with a serine, i.e., conservative amino acid replacements. Insertions or deletions may optionally be in the range of 1 to 5 amino acids. The variation allowed may be determined by systematically making insertions, deletions or substitutions of amino acids in the sequence and, if desired, testing the resulting variants for activity in assays known in the art or as described herein.

One embodiment of the invention is directed to ARTS variants which are fragments of the full length ARTS. Preferably, such fragments retain a desired activity or property of the full length ARTS protein. More preferably, the fragments retain the entire 27 amino acid C-terminus and the P-loop motif.

The variations can be made using methods known in the art such as oligonucleotide-mediated (site-directed) mutagenesis, alanine scanning, and PCR mutagenesis. Site-directed mutagenesis (Carter et al., Nucl. Acids Res., 13: 4331 (1986); Zoller et al., Nucl. Acids Res., 10: 6487 (1987)), cassette mutagenesis (Wells et al., Gene, 34: 315 (1985)), restriction selection mutagenesis (Wells et al., Philos. Trans. R. Soc. London SerA, 317: 415 (1986)) or other known techniques can be performed on the cloned DNA to produce the ARTS variant DNA.

B. Deletion Construct

A C-terminal deletion construct lacking the last 24 amino acids of the ARTS protein was constructed and its apoptotic activity in COS cells was examined. Within Within 1 hour of TGF-β treatment, this construct is found in the nucleus of transfected cells, and induces more than two fold increase in percent transfected apoptotic cells (52%), as compared with cells transfected with full length ARTS (18%). Twenty-four hours after TGF-β treatment both the full length and the truncated construct show similar percentage of apoptosis. Thus, the truncated construct is both more rapid in translocating to the nucleus and in inducing apoptosis in response to TGF-β.

An N- terminal deleted ARTS construct lacking the first 19 amino acids does not translocate to the nucleus in response to treatment with TGF-β. Moreover, co-localization experiments using the mitochondrial marker Minotracker red show that it does not localize to the mitochondria like the full lenghth protein. Consistent with the mislocalization, the N-terminally deleted ARTS construct does not induce apoptosis in cells treated with TGF-β. These data suggest that the N-terminal sequence of ARTS is important to its apoptogenic activity and its pattern of cellular localization.

C. Chimeric Proteins

The ARTS protein of the present invention may also be modified in a way to form a chimeric molecule comprising ARTS fused to another, heterologous polypeptide or amino acid sequence.

In one embodiment, such a chimeric molecule comprises a fusion of the ARTS with a tag polypeptide that provides an epitope to which an anti-tag antibody can selectively bind. The epitope tag is generally placed at the amino- or carboxyl-terminus of the protein. The presence of such epitope-tagged forms of the ARTS protein can be detected using an antibody against the tag polypeptide. Also, provision of the epitope tag enables the ARTS protein to be readily purified by affinity purification using an anti-tag antibody or another type of affinity matrix that binds to the epitope tag. Various tag polypeptides and their respective antibodies are well known in the art. Examples include poly-histidine (poly-his) or poly-histidine-glycine (poly-his-gly) tags; the flu HA tag polypeptide and its antibody 12CA5 (Field et al., Mol. Cell. Biol., 8: 2159-2165 (1988)); the c-myc tag and the 8F9, 3C7, 6E10, G4, B7 and 9E10 antibodies thereto (Evan et al., Molecular and Cellular Biology, 5: 3610-3616 (1985)); and the Herpes Simplex virus glycoprotein D (gD) tag and its antibody (Paborsky et al., Protein Engineering, 3(6): 547-553 (1990)). Other tag polypeptides include the Flag-peptide (Hopp et al., BioTechnology, 6: 1204-1210 (1988)); the KT3 epitope peptide (Martin et al., Science, 255: 192-194 (1992)); a tubulin epitope peptide (Skinner et al., J. Biol. Chem., 266:15163-15166 (1991)); and the T7 gene 10 protein peptide tag (Lutz-Freyermuth et al., Proc. Natl. Acad. Sci. USA, 87: 6393-6397 (1990)).

In an alternative embodiment, the chimeric molecule may comprise a fusion of the ARTS protein with an immunoglobulin or a particular region of an immunoglobulin. See, for example, LaRochelle et al., J. Cell Biol., 139(2): 357-66 (1995); Heidaran et al., FASEB J., 9(1): 140-5 (1995); Ashkenazi et al., Int. Rev. Immunol., 10(2-3): 219-27 (1993) and Cheon et al., PNAS USA, 91(3): 989-93 (1994).

D. Purification of ARTS

Forms of ARTS may be recovered from culture medium or from host cell lysates. If membrane-bound, it can be released from the membrane using a suitable detergent solution (e.g. Triton-X 100) or by enzymatic cleavage. Cells employed in expression of ARTS can be disrupted by various physical or chemical means, such as freeze-thaw cycling, sonication, mechanical disruption, or cell lysing agents.

It may be desired to purify ARTS from recombinant cell proteins or polypeptides. The following procedures are exemplary of suitable purification procedures: by fractionation on an ion-exchange column; ethanol precipitation; reverse phase HPLC; chromatography on silica or on a cation-exchange resin such as DEAE; chromatofocusing; SDS-PAGE; ammonium sulfate precipitation; gel filtration using, for example, Sephadex G-75; protein A Sepharose columns to remove contaminants such as IgG; and metal chelating columns to bind epitope-tagged forms of the ARTS. Various methods of protein purification may be employed and such methods are known in the art and described for example in Deutscher, Methods in Enzymology, 182 (1990); Scopes, Protein Purification: Principles and Practice, Springer-Verlag, New York (1982). The purification step(s) selected will depend, for example, on the nature of the production process used and the particular ARTS produced.

E. Production of ARTS

ARTS protein can be produced by culturing cells transformed or transfected with a vector containing a nucleic acid encoding ARTS. ARTS, or portions thereof, may also be produced by direct peptide synthesis using solid-phase techniques (see, e.g., Stewart et al., Solid-Phase Peptide Synthesis, W. H. Freeman Co., San Francisco, Calif. (1969); Merrifield, J. Am. Chem. Soc., 85: 2149-2154 (1963)). In vitro protein synthesis may be performed using manual techniques or by automation. Automated synthesis may be accomplished, for instance, using an Applied Biosystems Peptide Synthesizer (Foster City, Calif.) using manufacturer's instructions. Various portions of the ARTS protein may be chemically synthesized separately and combined using chemical or enzymatic methods to produce the full-length ARTS.

Host cells are transfected or transformed with expression or cloning vectors described herein for ARTS production and cultured in conventional nutrient media modified as appropriate for inducing promoters, selecting transformants, or amplifying the genes encoding the desired sequences. Culture conditions, such as media, temperature, and pH, can be selected by the skilled artisan without undue experimentation. In general, principles, protocols, and practical techniques for maximizing the productivity of cell cultures can be found in Mammalian Cell Biotechnology: a Practical Approach, M. Butler, ed. (IRL Press, 1991) and Sambrook et al., supra.

Methods of transfection are known, for example, CaPO₄ and electroporation. Depending on the host cell used, transformation is performed using standard techniques appropriate to such cells. The calcium treatment employing calcium chloride, as described in Sambrook et al., supra, or electroporation is generally used for prokaryotes or other cells that contain substantial cell-wall barriers. Infection with Agrobacterium tumefaciens is used for transformation of certain plant cells, as described by Shaw et al., Gene, 23: 315 (1983) and WO 89/05859 published Jun. 29, 1989. For mammalian cells without such cell walls, the calcium phosphate precipitation method of Graham and van der Eb, Virology, 52: 456-457 (1978) can be employed. General aspects of mammalian cell host system transformations have been described in U.S. Pat. No. 4,399,216. Transformations into yeast are typically carried out according to the method of Van Solingen et al., J. Bact., 130: 946 (1977) and Hsiao et al., Proc. Natl. Acad. Sci. (USA), 76: 3829 (1979). However, other methods for introducing DNA into cells, such as by nuclear microinjection, electroporation, bacterial protoplast fusion with intact cells, or polycations, e.g., polybrene, polyornithine, may also be used. For various techniques for transforming mammalian cells, see Keown et al., Methods in Enzymology, 185: 527-537 (1990) and Mansour et al., Nature, 336: 348-352 (1988).

Suitable host cells for cloning or expressing the DNA in the vectors herein include prokaryote, yeast, or higher eukaryote cells. Suitable prokaryotes include but are not limited to eubacteria, such as Gram-negative or Gram-positive organisms, for example, Enterobacteriaceac such as E. coli. Various E. coli strains are publicly available, such as E. coli-K12 strain MM294 (ATCC 31,446); E. coli X1776 (ATCC 31,537); E. coli strain W3110 (ATCC 27,325) and K5 772 (ATCC 53,635).

In addition to prokaryotes, eukaryotic microbes such as filamentous fungi or yeast are suitable cloning or expression hosts for ARTS-encoding vectors. Saccharomyces cerevisiae is a commonly used lower eukaryotic host microorganism.

Examples of invertebrate cells include insect cells such as Drosophila S2 and Spodoptera Sf9, as well as plant cells. Examples of useful mammalian host cell lines include Chinese hamster ovary (CHO) and COS cells. More specific examples include monkey kidney CV1 line transformed by SV40 (COS-7, ATCC CRL 1651); human embryonic kidney line (293 or 293 cells subcloned for growth in suspension culture, Graham et al., J. Gen Virol., 36: 59 (1977)); Chinese hamster ovary cells/DHFR (CHO, Urlaub and Chasin, Proc. Natl. Acad. Sci. USA, 77: 4216 (1980)); mouse sertoli cells (TM4, Mather, Biol. Reprod., 23: 243-251 (1980)); human lung cells (W138, ATCC CCL 75); human liver cells (Hep G2, HB 8065); and mouse mammary tumor (MMT 060562, ATCC CCL51). The selection of the appropriate host cell is deemed to be within the skill in the art.

Both expression and cloning vectors contain a nucleic acid sequence that enables the vector to replicate in one or more selected host cells. Such sequences are well known for a variety of bacteria, yeast, and viruses.

Expression and cloning vectors will typically contain a selection gene, also termed a selectable marker. Typical selection genes encode proteins that (a) confer resistance to antibiotics or other toxins, e.g., ampicillin, neomycin, methotrexate, or tetracycline, (b) complement auxotrophic deficiencies, or (c) supply critical nutrients not available from complex media, e.g., the gene encoding D-alanine racemase for Bacilli.

Expression and cloning vectors usually contain a promoter operably linked to the ARTS-encoding nucleic acid sequence to direct mRNA synthesis. Promoters recognized by a variety of potential host cells are well known.

Transcription of DNA encoding ARTS by higher eukaryotes may be increased by inserting an enhancer sequence into the vector. Enhancers are cis-acting elements of DNA, usually about from 10 to 300 bp that act on a promoter to increase its transcription. Many enhancer sequences are known.

Expression vectors used in eukaryotic host cells (yeast, fungi, insect, plant, animal, human, or nucleated cells from other multicellular organisms) will also contain sequences necessary for the termination of transcription and for stabilizing the mRNA. Such sequences are commonly available from the 5′ and, occasionally 3′, untranslated regions of eukaryotic or viral DNAs or cDNAs. These regions contain nucleotide segments transcribed as polyadenylated fragments in the untranslated portion of the mRNA encoding ARTS.

F. Gene Expression

Gene amplification and/or expression may be measured in a sample directly, for example, by conventional Southern blotting, Northern blotting to quantitate the transcription of mRNA (Thomas, Proc. Natl. Acad. Sci. USA, 77: 5201-5205 (1980)), dot blotting (DNA analysis), or in situ hybridization, using an appropriately labeled probe, based on the sequences provided herein. Alternatively, antibodies may be employed that can recognize specific duplexes, including DNA duplexes, RNA duplexes, and DNA-RNA hybrid duplexes or DNA-protein duplexes. The antibodies in turn may be labeled and the assay may be carried out where the duplex is bound to a surface, so that upon the formation of duplex on the surface, the presence of antibody bound to the duplex can be detected.

Gene expression, alternatively, may be measured by immunological methods, such as immunohistochemical staining of cells or tissue sections and assay of cell culture or body fluids, to quantitate directly the expression of gene product. Antibodies useful for immunohistochemical staining and/or assay of sample fluids may be either monoclonal or polyclonal and may be prepared in any mammal. Conveniently, the antibodies may be prepared against a native sequence ARTS polypeptide or against a synthetic peptide based on the DNA sequences provided herein or against exogenous sequence fused to ARTS DNA and encoding a specific antibody epitope.

VI. Domains

A. P-Loop Motif

The ARTS protein contains a P-loop motif (shown in bold italics in Table 2). Generally, the consensus sequence of a P-loop motif can be represented by the following: GXSGXGKS/T (SEQ. ID. NO: 4) (Aravind et al., (1999) Trends Biochem. Sci. 24: 47; and Saraste et al., (1990) Trends Biochem. Sci. 11: 430). The “X” in the consensus sequence represents any amino acid, including any of the 20 natural amino acids, or a modified or synthetic amino acid. The designation “S/T” means that the amino acid in this position can be either Serine or Threonine. It is important that the variant of the ARTS protein maintain a functional P-loop motif (e.g., fall under the consensus sequence). Without a functional P-loop motif, the protein is inactive and may even act as a dominant negative, blocking the activity of endogenous ARTS.

B. C-Terminal Sequence

The 27 amino acid C-terminus of ARTS (GPSLRLLAPPGAVKGTGQEHQGQGCH; SEQ. ID. NO: 5) appears to play a role in the cellular localization of ARTS. For example, C-terminal truncation of the ARTS amino acid sequence results in constitutive nuclear localization.

C. N-Terminal Sequence

The N-terminal sequence appears to function in the localization of ARTS to the mitochondria. Generally, N-terminal deletion mutants of ARTS are unable to localize to the mitochondria or to translocate to the nucleus following treatment with TGF-β.

VII. Antibody

The present invention further provides anti-ARTS antibodies. Exemplary antibodies include polyclonal, monoclonal, humanized, bispecific, and heteroconjugate antibodies.

The term “antibody” is used in the broadest sense and specifically covers single anti-ATRS monoclonal antibodies (including agonist, antagonist, and neutralizing antibodies) and anti-ARTS antibody compositions with polyepitopic specificity. The term “monoclonal antibody” as used herein refers to an antibody obtained from a population of substantially homogeneous antibodies, i.e., the individual antibodies comprising the population are identical except for possible naturally-occurring mutations that may be present in minor amounts.

If desired, the antibody can include a label so that the presence of ARTS can be detected and/or localized within a cell. Many suitable labels are known to those of skill in the art and include, for example, radiolabels (e.g., ³H, ¹⁴C, ³²P, ¹²⁵I, etc.), fluorescent labels (e.g., fluorescein, rhodamine, etc. ) or the use of enzyme conjugates (e.g., alkaline phosphatase, horseradish peroxidase, etc.) coupled with a chromogenic agent (e.g., 4chloro-1-napthol, 3,3′diaminobenzidine, 3,3′,5,5′-tetramethylbenzidine, etc.).

A. Polyclonal Antibodies

The anti-ARTS antibodies may comprise polyclonal antibodies. Methods of preparing polyclonal antibodies are known. Polyclonal antibodies can be raised in a mammal, for example, by one or more injections of an immunizing agent and, if desired, an adjuvant. Typically, the immunizing agent and/or adjuvant will be injected in the mammal by multiple subcutaneous or intraperitoneal injections. The immunizing agent may include the ARTS polypeptide or a fusion protein thereof. It may be useful to conjugate the immunizing agent to a protein known to be immunogenic in the mammal being immunized. Examples of such immunogenic proteins include but are not limited to keyhole limpet hemocyanin, serum albumin, bovine thyroglobulin, and soybean trypsin inhibitor. Examples of adjuvants that may be employed include Freund's complete adjuvant and MPL-TDM adjuvant (monophosphoryl Lipid A, synthetic trehalose dicorynomycolate). The immunization protocol may be selected by one skilled in the art without undue experimentation.

B. Monoclonal Antibodies

The anti-ARTS antibodies may, alternatively, be monoclonal antibodies. Monoclonal antibodies may be prepared using hybridoma methods, such as those described by Kohler and Milstein, Nature, 256: 495 (1975). In a hybridoma method, a mouse, hamster, or other appropriate host animal, is typically immunized with an immunizing agent to elicit lymphocytes that produce or are capable of producing antibodies that will specifically bind to the immunizing agent. Alternatively, the lymphocytes may be immunized in vitro.

The immunizing agent will typically include the ARTS polypeptide or a fusion protein thereof. Generally, either peripheral blood lymphocytes (“PBLs”) are used if cells of human origin are desired, or spleen cells or lymph node cells are used if non-human mammalian sources are desired. The lymphocytes are then fused with an immortalized cell line using a suitable fusing agent, such as polyethylene glycol, to form a hybridoma cell (Goding, Monoclonal Antibodies: Principles and Practice, Academic Press, (1986) pp. 59-103). Immortalized cell lines are usually transformed mammalian cells, particularly myeloma cells of rodent, bovine and human origin. Usually, rat or mouse myeloma cell lines are employed. The hybridoma cells may be cultured in a suitable culture medium that preferably contains one or more substances that inhibit the growth or survival of the unfused, immortalized cells. For example, if the parental cells lack the enzyme hypoxanthine guanine phosphoribosyl transferase (HGPRT or HPRT), the culture medium for the hybridomas typically will include hypoxanthine, aminopterin, and thymidine (“HAT medium”), which substances prevent the growth of HGPRT-deficient cells.

Preferred immortalized cell lines are those that fuse efficiently, support stable high level expression of antibody by the selected antibody-producing cells, and are sensitive to a medium such as HAT medium. More preferred immortalized cell lines are murine myeloma lines, which can be obtained, for instance, from the Salk Institute Cell Distribution Center, San Diego, Calif. and the American Type Culture Collection, Manassas, Va. Human myeloma and mouse-human heteromyeloma cell lines also have been described for the production of human monoclonal antibodies (Kozbor, J. Immunol., 133: 3001 (1984); Brodeur et al., Monoclonal Antibody Production Techniques and Applications, Marcel Dekker, Inc., New York, (1987) pp. 51-63).

The culture medium in which the hybridoma cells are cultured can then be assayed for the presence of monoclonal antibodies directed against ARTS. Preferably, the binding specificity of monoclonal antibodies produced by the hybridoma cells is determined by immunoprecipitation or by an in vitro binding assay, such as radioimmunoassay (RIA) or enzyme-linked immunoabsorbent assay (ELISA). Such techniques and assays are known in the art. The binding affinity of the monoclonal antibody can, for example, be determined by the Scatchard analysis of Munson and Pollard, Anal. Biochem., 107: 220 (1980).

After the desired hybridoma cells are identified, the clones may be subcloned by limiting dilution procedures and grown by standard methods (Goding, supra). Suitable culture media for this purpose include, for example, Dulbecco's Modified Eagle's Medium and RPMI-1640 medium. Alternatively, the hybridoma cells may be grown in vivo as ascites in a mammal.

The monoclonal antibodies secreted by the subclones may be isolated or purified from the culture medium or ascites fluid by conventional immunoglobulin purification procedures such as, for example, protein A-Sepharose, hydroxylapatite chromatography, gel electrophoresis, dialysis, or affinity chromatography.

The monoclonal antibodies may also be made by known recombinant DNA methods. DNA encoding the monoclonal antibodies of the invention can be readily isolated and sequenced using conventional procedures (e.g., by using oligonucleotide probes that are capable of binding specifically to genes encoding the heavy and light chains of murine antibodies). Once isolated, the DNA may be placed into expression vectors, which are then transfected into host cells such as simian COS cells, Chinese hamster ovary (CHO) cells, or myeloma cells that do not otherwise produce immunoglobulin protein, to obtain the synthesis of monoclonal antibodies in the recombinant host cells. The DNA also may be modified, for example, by substituting the coding sequence for human heavy and light chain constant domains in place of the homologous murine sequences or by covalently joining to the immunoglobulin coding sequence all or part of the coding sequence for a non-immunoglobulin polypeptide. Such a non-immunoglobulin polypeptide can be substituted for the constant domains of an antibody of the invention, or can be substituted for the variable domains of one antigen-combining site of an antibody of the invention to create a chimeric bivalent antibody.

C. Monovalent Antibodies

The antibodies may be monovalent antibodies. Methods for preparing monovalent antibodies are known. For example, one method involves recombinant expression of immunoglobulin light chain and modified heavy chain. The heavy chain is truncated generally at any point in the Fc region so as to prevent heavy chain crosslinking. Alternatively, the relevant cysteine residues are substituted with another amino acid residue or are deleted so as to prevent crosslinking.

In vitro methods are also suitable for preparing monovalent antibodies. Digestion of antibodies to produce fragments thereof, particularly, Fab fragments, can be accomplished using routine techniques known in the art.

D. Human and Humanized Antibodies

The anti-ARTS antibodies of the invention may further comprise humanized antibodies or human antibodies. Humanized forms of non-human (e.g., murine) antibodies are chimeric immunoglobulins, immunoglobulin chains or fragments thereof (such as Fv, Fab, Fab′, F(ab′)₂ or other antigen-binding subsequences of antibodies) which contain minimal sequence derived from non-human immunoglobulin. Humanized antibodies include human immunoglobulins (recipient antibody) in which residues from a complementary determining region (CDR) of the recipient are replaced by residues from a CDR of a non-human species (donor antibody) such as mouse, rat or rabbit having the desired specificity, affinity and capacity. In some instances, Fv framework residues of the human immunoglobulin are replaced by corresponding non-human residues. Humanized antibodies may also comprise residues that are found neither in the recipient antibody nor in the imported CDR or framework sequences. In general, the humanized antibody will comprise substantially all of at least one, and typically two, variable domains, in which all or substantially all of the CDR regions correspond to those of a non-human immunoglobulin and all or substantially all of the FR regions are those of a human immunoglobulin consensus sequence. The humanized antibody optimally also will comprise at least a portion of an immunoglobulin constant region (Fc), typically that of a human immunoglobulin (Jones et al., Nature, 321: 522-525 (1986); Riechmann et al., Nature, 332: 323-329 (1988); and Presta, Curr. Op. Struct. Biol., 2: 593-596 (1992)).

Methods for humanizing non-human antibodies are known. Generally, a humanized antibody has one or more amino acid residues introduced into it from a source that is non-human. Typically, humanized antibodies are human antibodies in which some CDR residues and possibly some FR residues are substituted by residues from analogous sites in rodent antibodies. Other methods for generating human and humanized antibodies are known.

E. Bispecific Antibodies

Bispecific antibodies are monoclonal, preferably human or humanized, antibodies that have binding specificities for at least two different antigens. In the present case, one of the binding specificities is for the ARTS, the other one is for any other antigen, and preferably for a cell-surface protein or receptor or receptor subunit.

Methods for making bispecific antibodies are known. Traditionally, the recombinant production of bispecific antibodies is based on the co-expression of two immunoglobulin heavy-chain/light-chain pairs, where the two heavy chains have different specificities (Milstein and Cuello, Nature, 305: 537-539 (1983)). Because of the random assortment of immunoglobulin heavy and light chains, these hybridomas (quadromas) produce a potential mixture of ten different antibody molecules, of which only one has the correct bispecific structure. Purification of the correct molecule is usually accomplished by affinity chromatography.

Antibody variable domains with the desired binding specificities (antibody-antigen combining sites) can be fused to immunoglobulin constant domain sequences. The fusion preferably is with an immunoglobulin heavy-chain constant domain, comprising at least part of the hinge, CH2, and CH3 regions. It is preferred to have the first heavy-chain constant region (CH1) containing the site necessary for light-chain binding present in at least one of the fusions. DNAs encoding the immunoglobulin heavy-chain fusions and, if desired, the immunoglobulin light chain, are inserted into separate expression vectors, and are co-transfected into a suitable host organism. For further details of generating bispecific antibodies see, for example, Suresh et al., Methods in Enzymology, 121: 210 (1986).

F. Heteroconjugate Antibodies

Heteroconjugate antibodies are also within the scope of the present invention. Heteroconjugate antibodies are composed of two covalently joined antibodies. It is contemplated that the antibodies may be prepared in vitro using known methods in synthetic protein chemistry, including those involving crosslinking agents. For example, immunotoxins may be constructed using a disulfide exchange reaction or by forming a thioether bond. Examples of suitable reagents for this purpose include iminothiolate and methyl-4-mercaptobutyrimidate.

G. Uses for anti-ARTS Antibodies

The anti-ARTS antibodies of the invention have various utilities. For example, anti-ARTS antibodies may be used in diagnostic assays for ARTS, e.g., detecting its expression in specific cells or tissues. Various diagnostic assay techniques known in the art may be used, such as competitive binding assays, direct or indirect sandwich assays and immunoprecipitation assays conducted in either heterogeneous or homogeneous phases (Zola, Monoclonal Antibodies: A Manual of Techniques, CRC Press, Inc. (1987) pp. 147-158). The antibodies used in the diagnostic assays can be labeled with a detectable moiety. The detectable moiety should be capable of producing, either directly or indirectly, a detectable signal. For example, the detectable moiety may be a radioisotope, such as ³H, ¹⁴C, ³²P, ³⁵S, or ¹²⁵I, a fluorescent or chemiluminescent compound, such as fluorescein isothiocyanate, rhodamine, or luciferin, or an enzyme, such as alkaline phosphatase, beta-galactosidase or horseradish peroxidase. Any method known in the art for conjugating the antibody to the detectable moiety may be employed, including those methods described by Hunter et al., Nature, 144: 945 (1962); David et al., Biochemistry, 13: 1014 (1974); Pain et al., J. Immunol. Meth., 40: 219 (1981); and Nygren, J. Histochem. and Cytochem., 30: 407 (1982).

Anti-ARTS antibodies also are useful for the affinity purification of ARTS from recombinant cell culture or natural sources. In this process, the antibodies against ARTS are immobilized on a suitable support, such a Sephadex resin or filter paper, using methods well known in the art. The immobilized antibody then is contacted with a sample containing the ARTS to be purified, and thereafter the support is washed with a suitable solvent that will remove substantially all the material in the sample except the ARTS, which is bound to the immobilized antibody. Finally, the support is washed with another suitable solvent that will release the ARTS from the antibody.

VIII. Uses

The nucleic acid sequence encoding ARTS, or the ARTS protein itself can be used to increase or decrease the apoptotic response of a cell.

A. Method to Increase Sensitivity of a Cell to Apoptosis

According to one embodiment, the sensitivity of a cell to apoptosis (induced by TGF-β1 is increased by administering ARTS protein to a cell. As used herein, the term “administering” refers to a process wherein ARTS protein itself is administered to a cell or wherein a nucleic acid sequence encoding ARTS is administered to a cell. For example, the sensitivity of a tumor cell to TGF-β induced apoptosis can be restored by administering ARTS protein to the cell or by transfecting the cell with a nucleic acid sequence encoding ARTS. Examples of tumor cells that are not sensitive to TGF-β induced apoptosis include COS cells (monkey kidney transformed cells), and Hela cells (human cervix adenocarcinoma). Administration of ARTS or transfection of ARTS into either of these cell lines makes them competent to undergo apoptosis in response to treatment with TGF-β.

B. Method to Decrease Sensitivity of a Cell to Apoptosis

It appears that expression of ARTS is necessary to direct TGF-β induced apoptosis. Thus, transfection of cells with anti-sense ARTS can reduce apoptotic response to TGF-β.

C. Transgenic animals

Nucleic acids that encode ARTS or its modified forms can also be used to generate either transgenic animals or “knock out” animals which, in turn, are useful in the development and screening of therapeutically useful reagents. A transgenic animal (e.g., a mouse or rat) is an animal having cells that contain a transgene, which transgene was introduced into the animal or an ancestor of the animal at a prenatal, e.g., an embryonic stage. A transgene is a DNA that is integrated into the genome of a cell from which a transgenic animal develops. In one embodiment, cDNA encoding ARTS can be used to clone genomic DNA encoding ARTS in accordance with established techniques and the genomic sequences used to generate transgenic animals that contain cells which express DNA encoding ARTS. Methods for generating transgenic animals, particularly animals such as mice or rats, are conventional in the art. Typically, particular cells would be targeted for ARTS transgene incorporation with tissue-specific enhancers. Transgenic animals that include a copy of a transgene encoding ARTS introduced into the germ line of the animal at an embryonic stage can be used to examine the effect of increased expression of DNA encoding ARTS. Such animals can be used as tester animals for reagents thought to confer protection from, for example, pathological conditions associated with its overexpression or underexpression. In accordance with this facet of the invention, an animal is treated with the reagent and a reduced incidence of the pathological condition, compared to untreated animals bearing the transgene, would indicate a potential therapeutic intervention for the pathological condition.

Alternatively, non-human homologues of ARTS can be used to construct a ARTS “knock out” animal which has a defective or altered gene encoding ARTS as a result of homologous recombination between the endogenous gene encoding ARTS and altered genomic DNA encoding ARTS introduced into an embryonic cell of the animal. For example, cDNA encoding ARTS can be used to clone genomic DNA encoding ARTS in accordance with established techniques. A portion of the genomic DNA encoding ARTS can be deleted or replaced with another gene, such as a gene encoding a selectable marker that can be used to monitor integration. Typically, several kilobases of unaltered flanking DNA (both at the 5′ and 3′ ends) are included in the vector (see e.g., Thomas and Capecchi, Cell, 51: 503 (1987) for a description of homologous recombination vectors). The vector is introduced into an embryonic stem cell line (e.g., by electroporation) and cells in which the introduced DNA has homologously recombined with the endogenous DNA are selected (see e.g., Li et al., Cell, 69: 915 (1992)). The selected cells are then injected into a blastocyst of an animal (e.g., a mouse or rat) to form aggregation chimeras (see e.g., Bradley, in Teratocarcinomas and Embryonic Stem Cells: A Practical Approach, E. J. Robertson, ed. (IRL, Oxford, 1987), pp. 113-152). A chimeric embryo can then be implanted into a suitable pseudopregnant female foster animal and the embryo brought to term to create a “knock out” animal. Progeny harboring the homologously recombined DNA in their germ cells can be identified by standard techniques and used to breed animals in which all cells of the animal contain the homologously recombined DNA. Knockout animals can be characterized for instance, for their ability to defend against certain pathological conditions and for their development of pathological conditions due to absence of the ARTS polypeptide.

D. Gene Therapy

Nucleic acid encoding the ARTS polypeptide may also be used in gene therapy. In gene therapy applications, genes are introduced into cells in order to achieve in vivo synthesis of a therapeutically effective genetic product, for example for replacement of a defective gene. “Gene therapy” includes both conventional gene therapy where a lasting effect is achieved by a single treatment, and the administration of gene therapeutic agents, which involves the one time or repeated administration of a therapeutically effective DNA or mRNA. Antisense RNAs and DNAs can be used as therapeutic agents for blocking the expression of certain genes in vivo. It has already been shown that short antisense oligonucleotides can be imported into cells where they act as inhibitors, despite their low intracellular concentrations caused by their restricted uptake by the cell membrane. (Zamecnik et al., Proc. Natl. Acad. Sci. USA 83, 4143-4146 (1986)). The oligonucleotides can be modified to enhance their uptake, e.g. by substituting their negatively charged phosphodiester groups by uncharged groups.

There are a variety of techniques available for introducing nucleic acids into viable cells. The techniques vary depending upon whether the nucleic acid is transferred into cultured cells in vitro, or in vivo in the cells of the intended host. Techniques suitable for the transfer of nucleic acid into mammalian cells in vitro include the use of liposomes, electroporation, microinjection, cell fusion, DEAE-dextran, the calcium phosphate precipitation method, etc. The currently preferred in vivo gene transfer techniques include transfection with viral (typically retroviral) vectors and viral coat protein-liposome mediated transfection (Dzau et al., Trends in Biotechnology 11, 205-210 (1993)). In some situations it is desirable to provide the nucleic acid source with an agent that targets the target cells, such as an antibody specific for a cell surface membrane protein or the target cell, a ligand for a receptor on the target cell, etc. Where liposomes are employed, proteins which bind to a cell surface membrane protein associated with endocytosis may be used for targeting and/or to facilitate uptake, e.g. capsid proteins or fragments thereof tropic for a particular cell type, antibodies for proteins which undergo internalization in cycling, proteins that target intracellular localization and enhance intracellular half-life. The technique of receptor-mediated endocytosis is described, for example, by Wu el al., J. Biol. Chem. 262, 4429-4432 (1987); and Wagner et al., Proc. Natl. Acad. Sci. USA 87, 3410-3414 (1990). For review of gene marking and gene therapy protocols see Anderson et al., Science 256, 808-813 (1992).

It is believed that the ARTS gene therapy has applications in, for instance, treating cancerous tumors. This can be accomplished, for example, using the techniques described above and by introducing a viral vector containing a ARTS gene into certain tissues (like a tumor) to increase sensitivity to TGF-β induced apoptosis.

E. Diagnostics

ARTS may also be employed in diagnostic methods. For example, the presence or absence of ARTS or a nucleic acid sequence encoding a functional ARTS protein, mislocalization of ART, or alternatively over- or under-expression of ARTS in an individual's cells, can be detected. Many methods are known for detecting the presence of a protein or nucleic acid. For example, anti-ARTS antibodies can be used to detect the presence of ARTS protein. Complementary oligonucleotides can be used to detect the presence of a nucleic acid sequence encoding ARTS.

The skilled practitioner may use information resulting from such detection assays to assist in determining whether a cell is sensitive to TGF-β induced apoptosis.

Detection of impaired ARTS function in the mammal may also be used to assist in diagnosis. Preliminary studies on human astrocytoma sections have shown that these tumor cells contain large amounts of ARTS protein, in contrast to no ARTS protein or very little detected in normal astrocytes. In addition, large amounts of ARTS protein were found in sections of human Duchenne muscular dystrophy patients, while matching tissues from normal sections showed low staining for ARTS protein.

The invention also includes kits for detecting the presence of ARTS protein or a nucleic acid encoding ARTS in a sample. As used herein, the term “sample” generally refers to at least one cell (e.g., a single cell, a suspension of cells, or a tissue), or a cell lysate. However, the sample need not be obtained from a cell. For example, a kit for detecting the presence of ARTS protein may contain an antibody capable of immunoreacting with the protein or a nucleic acid sequence capable of hybridizing to a nucleic acid sequence encoding ARTS. If desired the antibody or the complementary nucleic acid sequence can include a label (e.g., a fluorescent label, radiolabel, enzyme conjugate, etc.)

### WORKING EXAMPLES

### Example I

### Retroviral Insertional Mutagenesis

Retroviral insertional mutagenesis of rat prostate carcinoma cells (NRP-154) was used to obtain mutant cell lines resistant to TGF-β1. NRP-154 cells were selected because this cell line has been previously shown to be sensitive to TGF-β induced apoptosis (Prehn et al., (1994) Proc. Natl. Acad. Sci U.S.A., 91: 12599-12603); Ren et al., (1997) Brain Res. Mol. Brain Res., 48: 315-322; Danielpour et al., (1994) Cancer Research, 54: 3413-3421; and Hsing et al., (1996) Cancer Res., 56: 5146-5149).

NRP-154 rat prostatic epithelial cells were derived from the non-neoplastic dorsal-lateral prostate of Lobund Wistar rats and treated with N-methyl-N-nitrosurea and testosterone propionate as described by Prehn et al., (1994) Proc. Natl. Acad. Sci. U.S.A., 91: 12599-12603. The NRP-154 cells are then grown in DMEM/F12 medium containing 10% fetal bovine serum and antibiotics in 5% CO₂ atmosphere. 5×10⁷ NRP-154 cells, cultured as described, were infected with the Moloney Leukemia virus vector PLNCX containing the neomycin resistance gene (NeO) (Saraste et al., (1990) Trends Biochem. Sci. 11: 430-434). The amphotropic line PA317 were used as packaging cells. Two infections of 5×10⁷ cells were performed.

### Example II

### Clone Isolation

Phenotypic selection was used to isolate clones resistant to TGF-β induced apoptosis. To select for cells mutated in the TGF-β signaling pathway, the mutant cells were cultured for 17 days in the presence of 20 ng/ml recombinant human TGF-β1 (R&D Systems, Inc., Minneapolis, Minn.). To eliminate spontaneous mutants, the cells were then cultured for an additional 21 days in the presence of both TGF-β1 and 100 μg/ml G418 (Geneticin, Gibco BRL Life Technologies, Gaithersburg, Md.). Fifteen clones resistant to both TGF-β1 and G418 were isolated. Three clones, M-NRP1, M-NRP2 and M-NRP3 were expanded and further characterized.

The table below provides a comparison of the parent NRP-154 cell line and one of the mutant clones, M-NRP1.

| Parent Cell: NRP-154 | Clone: M-NRP1 |
|---|---|
| Expresses TGF-β receptors | Expresses TGF-β receptors |
| Expression of target genes are | Expression of target genes are |
| induced in response to TGF-β | induced in response to TGF-β |
| Apoptotic response to Staurosporine | Apoptotic response to |
| and okadaic acid | Staurosporine and okadaic acid |
| Indicators of cell growth are strongly | Indicators of cell growth are |
| suppressed by TGF-β | unaffected by TGF-β |
| Sensitive to TGF-β induced apoptosis | Overexpression of anti-apoptotic |
| | protein BCL-XL |
| | Dysregulation of ARTS |
| | Not sensitive to TGF-β apoptosis |

### Example III

### Gene Expression

To ascertain whether the insensitivity of the mutant cells to TGF-β induced apoptosis was due to loss of receptor expression or signaling, pathways modulated by TGF-β1 were examined to determine whether signaling to these targets was disrupted.

A. Receptor Expression

To determine whether the TGF-β receptors were expressed in the three clones, a receptor cross-linking assay was performed.

Recombinant human TGF-β1 was labeled with ¹²⁵Iodine using the chloramine-T method (Kyprianou et al., (1989) Mol. Endocrinol. 3: 1515-1522). Cells were seeded at 60% confluence in 100-mm plates and incubated for 4 hours at 4° C. with 100 pM (¹²⁵I)TGF-β1 with or without 100-fold excess of unlabeled TGF-β 1. Cross-linking was performed with disuccinimidyl suberate as described by Guo et al., (1999) Cancer Res 59: 1366-1371. Samples were subjected to electrophoresis on a 4-12% gradient sodium dodecyl sulfate-polyacrylamide gel (SDS-PAGE) followed by autoradiography.

All three clones expressed all three types of TGF-β receptors: type I and type II signaling receptors and the higher molecular weight betaglycan, or type III receptor (Heldin et al., (1997) Nature, 390: 465-471). M-NRP1 cells expressed elevated levels of all three types of receptors as compared to the parental (NRP-154) cells, whereas expression levels in M-NRP2 and M-NRP3 cells were comparable to those of the parental cells. A549 cells were used as a positive control.

B. Receptor Signaling

To determine whether the TGF-β receptors on the mutant cell lines were still competent for signaling, the effects of TGF-β on gene induction was examined. Induction of plasminogen activator inhibitor-1 (PAI-1) and fibronectin was examined. Both PAI-1 and fibronectin are commonly assayed endpoints of TGF-β signaling (Roberts et al., (1990). The transforming growth factor-β. Handbook of Experimental Pharmacology: Peptide Growth Factors and Their Receptors, M. B. Sporn and A. B. Roberts, (eds), Springer-Verlag, pp. 419-472; Heldin et al., (1997) Namfe, 390: 465-471; Wrana et al., (1992) Cell, 71: 1003-1014; and Danielpour, D. (1996) J. Cell. Physiol. 166: 231-239).

To examine the protein levels of PAI-1 and fibronectin in NRP-154 and M-NRP1, M-NRP2 and M-NRP3 cell lines, cells were seeded in 6-well dishes and incubated for 18 hours in medium containing 0.2% fetal bovine serum with or without 10 ng/ml TGF-β1. The cells were then labeled with 50 μCi/ml (³⁵S)methionine for 6 hours.

After metabolic labeling, PAI-1 and fibronectin were extracted from the cell-associated extracellular matrix and supernatants, respectively, as described by Nass et al., (1996) Biochem. Biophys. Res. Commun., 227: 248-256). Both preparations were subjected to 10% SDS-polyacrylamide gel electrophoresis followed by autoradiography. PAI-1 and fibronectin were identified by their characteristic sizes (45 and 240 kDa, respectively) and inducibility by TGF-β.

Following TGF-β treatment, all three mutant cell lines secreted lower amounts of PAI-1 than the parental cells. In all three mutant cell lines, secretion of fibronectin was comparable to that of the NRP-154 cells. This data indicates that TGF-β is still capable of inducing the expression of these genes in the selected mutant cells, although the strength of the signal may differ from the parental cells. Thus, signaling pathways leading to expression of immediate gene targets of TGF-β are not interrupted in the mutant cells resistant to TGF-β-induced apoptosis.

C. TGF-β Inducibility of Reporter Constructs

The ability of TGF-β to stimulate the activity of two reporter constructs (p8001uc and the artificial p3TP-Lux reporter) was examined. Based on the level of receptor expression, M-NRP1 cells were chosen for this analysis and subsequent studies.

p8001uc is an 800 bp TGF-β responsive region of the PAI-1 gene promoter (Keeton et al., (1991) J. Biol. Chem. 266: 23048-23052). The p3TP-Lux reporter contains 3 repeats of an AP-1 site from the collagenase promoter linked to a short region of the PAI-1 promoter (Wrana et al., (1992) Cell, 71: 1003-1014).

NRP-154 cells and M-NRP1 cells were transiently transfected with p8001uc or p3TP-Lux reporter constructs using the Lipofectamine transfection reagent (Gibco BRL Life Technologies, Gaithersburg, Md.) Briefly, cells were seeded to 50% confluence in 6-well plates in DMEM-F12 containing 10% fetal calf serum and incubated at 37° C. overnight. Cells were transfected with the reporter constructs p3TP-Lux (Nass et al., (1996) Biochem. Biophys. Res. Commun., 227: 248-256), p8001uc (Danielpour et al., (1994) Cancer Research, 54: 3413-3421), or empty vector, according to the manufacturer's protocol. After 24 hours, the cells were washed, and allowed to recover for 24 hours in growth medium. After 24 hours, the medium was replaced with 1% FBS-containing medium, with or without 10 ng/ml TGF-β and the cells were incubated for further 24 hrs.

Luciferase activity was determined in the cell lysate using an assay kit (Analytic Luminescence Laboratory) and a luminometer (ML 3000; Dynatech Laboratories). Activities were normalized on the basis of β-galactosidase expression from pSV-galactosidase in all luciferase reporter experiments. All experiments were repeated at least three times with similar results. Results are expressed as the mean (+/−SEM) of three replicate assays.

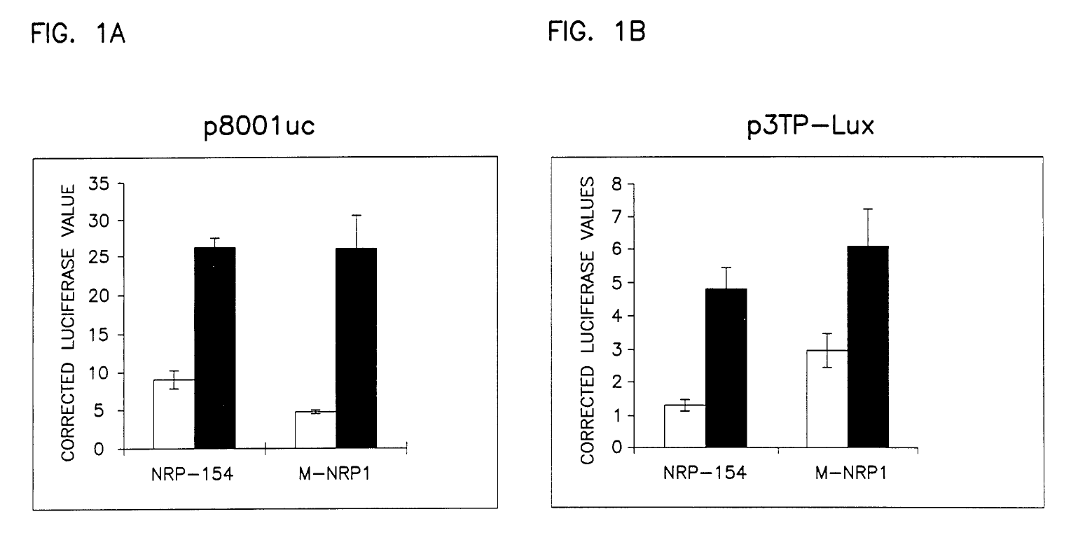

Transient transfection of cells with the reporter constructs showed that TGF-β inducibility remained intact in M-NRP1 cells compared to the NRP-154 cells (FIGS. 1A, B), consistent with the inducibility of PAI-1 protein. Thus, the ability of TGF-β to induce the activity of p8001uc and p3TP-Lux promoter constructs is similar in M-NRP1 and NRP-154 cells. In FIG. 1, cells treated with TGF-β are represented by shaded bars. Untreated cells are shown as open bars.

D. Conclusion

These experiments, which are based on both protein expression and on induction of promoter activity, demonstrate that interruption of the TGF-β apoptotic signaling pathway in the mutant clones did not affect early events leading to induction of direct gene targets such as PAI-1 and fibronectin. Because the pathways mediating activation of direct target genes such as induction of fibronectin expression, dependent on JNK activation (Hocevar et al., (1999) EMBO J. 18: 1345-1356), and induction of plasminogen activator inhibitor (PAI-1), dependent on a Smad signaling pathway (Stroschein et al., (1999) J. Biol. Chem., 274: 9431-9441; Song et al., (1998) J. Biol. Chem. 273: 29287-29290; and Hua et al, (1998) Genes Dev. 12: 3084-3095), were still functional in the mutant cells, receptor signaling to these targets appears to be, to a large degree, independent of the disrupted-apoptotic pathway.

### Example IV

### Morphology

M-NRP1 cells were distinguishable morphologically from the parental cells. The M-NRP1 cells appear as rounded piles of grape-like cells as compared to the more flattened cuboidal monolayer characteristic of the NRP-154 cells (data not shown). Interestingly, whereas the parental cells do not change morphologically in response to treatment with 10 ng/ml of TGF-β for 24 hours, M-NRP1 cells change shape dramatically under these same conditions and assume a more flattened morphology similar to the parental cells (data not shown).

### Example V

### Resistance to TGF-β Induced Apoptosis

M-NRP1 cells were tested for resistance to TGF-β induced apoptosis and apoptosis caused by other agents. M-NRP1 cells, obainted as described in Examples I and II, were cultured for five weeks in media that included 10 ng/ml TGF-β (R & D Systems, Inc., Minneapolis, Minn.) and G418 (Geneticin, Gibco BRL Life Technologies, Gaithersburg, Md.). After five weeks, the cells were isolated as described in Example II, above.

A. Detection of Apoptosis

2×10⁵ M-NRP1 cells or NRP-154 cells were plated in 6-well dishes. After 24 hours, 2 ml DMEM/F12 containing 15 mM HEPES, 1% calf serum, and 0.1 μM dexamethasone were added to the plates. The cells were cultured at 37° C. for 24 hours and then detached by trypsinization.

Internucleosomal DNA ladders were detected with a modification (described below) of TACS apoptotic DNA ladder kit (Trevigen, Githersburg, Md.). Briefly, the detached cells were centrifuged at 600×g to form cell pellets. The cell pellets were then resuspended in 25 μl PBS and lysed by the addition of 25 μl of lysis buffer. DNA was purified from the mixture as directed by Trevigen.

The nicked ends of the DNA were ³²P-labeled by incubating 1 μg of DNA with 2.5 units of Klenow fragment of DNA Poll in the presence of 0.5 μCi of (α-³²P) dCTP (3 Ci/mmol, Dupont NEN, Boston, Mass.) for 30 min at room temperature in a 10 μl reaction volume containing 5 mM MgCl₂ and 10 mM Tris-HCl (pH 7.5). One third of the labeled DNA was electrophoresed through 1.8% agarose-1×TAE at 70 V for 2 hours. Gels were dried and exposed to X-Omat AR (Kodak, Rochester, N.Y.) for about 1 hour.

Parental NRP-154 cells typically show over 80% cell death after 48-72 hours exposure to 10 ng/ml TGF-β (Ren et al., (1997) Mol. Brain Res., 48: 315-322). In contrast, the mutant clones isolated after five weeks of treatment with TGF-β followed by G418 were resistant to TGF-β induced apoptosis.

B. TUNEL Assay

To confirm the resistance of cloned M-NRP1 cells to TGF-β apoptosis, a TUNEL (Terminal Deoxynucleotidyl Transferase-Mediated Uridine Nick End Labeling) assay was performed on NRP-154 and M-NRP1 cells, cultured as described in Part A, above.

TUNEL assays were performed using the TACS In Situ Apoptosis Detection Kit (Trevigen). Briefly, NRP-154 and M-NRP1 cells were plated at 8×10⁴ cells/ml in a 96 well plate and allowed to attach overnight. Cells were then treated with either 10 ng/ml TGF-β (R&D Systems, Inc., Minneapolis, Minn.), or control (i.e., the identical solution without TGF-β) for 24 hours, fixed with formaldehyde and analyzed for stained nuclei according to the manufacturer's instructions.

NRP-154 cells show strong TUNEL staining of apoptotic nuclei after 24 hours treatment with TGF-β, whereas most nuclei from M-NRP1 cells remained intact (data not shown).

### Example V

### Other Apoptosis Inducing Agents

To determine whether the block in apoptosis was specific for pathways downstream from TGF-β, or whether the mutant cells had a block in general apoptotic mechanisms, M-NRP1 cells were tested for responsiveness to other apoptosis-inducing agents such as staurosporine and okadaic acid.

Staurosporine is a non-specific inhibitor of protein kinases that can induce apoptosis in nearly all cells (Bertrand et al., (1994) Cell Res, 211: 314-321; Krohm et al., (1998) J. Neurosci. 18: 8186-8197; and Yue et al., (1998) J. Mol. Cell. Cardiol. 30: 495-507). Okadaic acid is a microbial inhibitor of phosphoprotein phosphatases 1 and 2A and is known to induce apoptosis in many cell types (Benito et al., (1997) Leukemia 11: 940-944).

Staurosporine and okadaic acid (Upstate Biotechnology Incorporated, Lake Placid, N.Y.) were dissolved in dimethyl sulfoxide (DMSO) and diluted to a final concentration of 2 nM and 60 nM, respectively, in 0.01% DMSO.

NRP-154 and M-NRP1 cells were treated with a control (i.e., the identical solution without the agent of interest) or with 10 ng/ml TGF-β, okadaic acid (30 nM) or staurosporine (2 nM) for 24 hours. Apoptosis was determined by DNA laddering, essentially as described in Example VA, above.

Both parental and mutant cells exhibited morphologically evident shrinkage, nuclear fragmentation, and loss of attachment characteristic of apoptosis following treatment with staurosporine and okadaic acid (data not shown). Thus, whereas M-NRP1 cells are resistant to apoptosis induced by treatment with TGF-β, they remain sensitive to apoptosis induced by treatment with either okadaic acid or staurosporine, similar to parental NRP-154 cells.

This data demonstrates that the mutant cells have not lost the apoptotic mechanisms activated by treatment with staurosporine and okadaic acid, including caspase activation (Krohm et al., (1998) J. Neurosci. 18: 8186-8197; Yue et al., (1998) J. Mol. Cell. Cardiol. 30: 495-507; and Benito et al., (1997) Leukemia 11: 940-944). The mutant cells therefore show selective loss of the apoptotic pathways induced by TGF-β.

### Example VI

### Growth Inhibition

Apoptotic stimuli often arrest cell growth before inducing cell death. Thus, two experiments were performed to determine whether M-NRP1 cells were resistant to growth inhibition.

M-NRP1 and NRP-154 cells were cultured in 10% fetal bovine serum as described by Chaudhary et al., (1998) J. Biol. Chem. 273: 17708-17712) and exposed to 10 ng/ml TGF-β1 for 24 hours.

A. pCal-Luc

Suppression of the activity of pCAL2, a cyclin A reporter construct that includes a 760 bp TGF-β responsive region, can be used as a surrogate marker for growth inhibition (Feng et al., (1995) J. Biol. Chem. 270: 24237-4245.

M-NRP1 cells were transiently transfected with pCal-luc (a pCAL2 reporter construct described by Keeton et al., (1991) J. Biol. Chem. 266: 23048-23052) using the Lipofectamine (Gibco BRL Life Technologies, Gaithersburg, Md.) transfection reagent.

Activity of the transiently transfected cyclin A promoter construct, pCal2-luc, was assessed by a luciferase assay and measured using a luminometer. The basal activity (e.g., without TGF-β treatment) of pCAL2 was similar in MNRP-1 and NRP-154 cells.

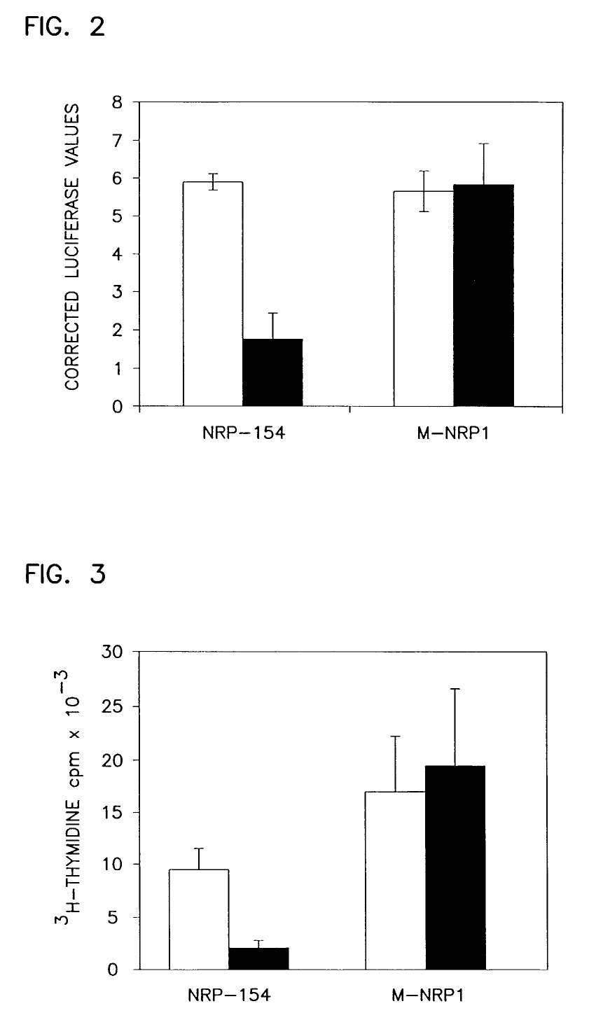

NPR-154 cells showed a strong decrease in pCAL2 activity after 24 hours of treatment with 10 ng/ml TGF-β. In contrast, the activity of the pCal-luc reporter was unaffected M-NRP1 cells after 24 hours of exposure to 10 ng/ml TGF-β. The results are shown in FIG. 2 (TGF-β treatment, dark bars; control, open bars). The results are corrected for transfection efficiency and are expressed as the mean (+/−SEM) of three repeated luciferase assays. Results are expressed as the mean (+/−SD) of 3 replicates from a representative experiment.

The reduction in activity is not due to cell loss, since TGF-β induced the activity of the p8001uc and 3tp-Lux reporters within the same time frame (See FIG. 1).

The lack of suppression of pCAL2 activity in M-NRP1 cells following TGF-β treatment indicates a coordinate loss of sensitivity to TGF-β mediated growth inhibition and apoptosis in these cells.

B. Thymidine Incorporation Assay

Thymidine uptake can be used as an indicator of the responsiveness of cells to apoptotic inducing agents (King et al., (1998) Annu. Rev. Physiol. 60: 601-617).

M-NRP1 and NRP-154 cells were plated at 1×10⁵ cells per well in 24 well plates and allowed to attach overnight. Incorporation of (³H)-thymidine was measured by adding radioactivity for 3 hours, 24 hours after addition of control or 10 ng/ml TGF-β1. Plates were counted on a Packard Top Count. Parallel plates (e.g., those treated in an identical manner) were harvested by trypinization for counting in a Coulter Counter.

As shown in FIG. 3, after 24 hr treatment with 10 ng/ml TGF-β1, (³H)-thymidine uptake in M-NRP1 cells was stimulated 14%, whereas that of NRP-154 cells was inhibited greater than 80%. These results demonstrate that M-NRP1 was insensitive to growth inhibition by TGF-β.

Treatment for 24 hours with 10 ng/ml TGF-β is shown by closed bars. Treatment with control is shown by open bars. Results are expressed as the mean (+/−SD) of 3 replicates from a representative experiment of 4.

### Example VII

### Expression of Bcl-xL

To gain insight into possible mechanisms underlying the resistance of the M-NRP1 cells to TGF-β induced apoptosis, the expression of the Bcl-2 family of apoptotic regulatory proteins was investigated.

The Bcl-2 family of apoptotic regulatory proteins are believed to have a role in TGF-β mediated apoptosis (Saltzman et al., (1998) Exp. Cell Res., 242: 244-254; Prehn et al., (1994) Proc. Natl. Acad. Sci. U.S.A., 91: 12599-12603; Nass et al., (1996) Biochem. Biophys. Res. Commun., 227: 248-256).

A. Northern Blot Analysis

Northern blot analysis was used to examine mRNA levels of Bcl-2, Bcl-xL and Bax in NRP-154 and M-NRP1 cell lines in response to treatment with TGF-β.

Cell lysates were prepared (as described below) from confluent NRP-154 and M-NRP1 cultures, with and without treatment with TGF-β1 (10 ng/ml) for 48 hours. Briefly, the cells were rinsed with phosphate-buffered saline and transferred to 0.5 ml of ice-cold lysis buffer (phosphate-buffered saline, 1% Nonidet P-40, 0.5% sodium deoxycholate, 0.1% SDS) containing protease and phosphatase inhibitors (50 μg/ml phenylmethylsulfonyl fluoride, 30 μg/ml aprotinin, 5 μg/ml leupeptin, 30 100 mM sodium orthovanadate). The mixture was passed several times through a 21-gauge needle. The cell lysates were then incubated on ice for 30 min and microfuged for 20 min at 4° C.

Northern blots were performed as described by Nass et al., (1996) Biochem. Biophys. Res. Commun., 227: 248-256. Briefly, cellular mRNA was fractionated on 1% formaldehyde-agarose gels and transferred to nylon membrane (Hybond-N, Amersham Corp.). The filters were probed with actin, or Bax, Bcl-2 and Bcl-xL DNA fragments generated by RT-PCR and labeled with (³²P)dCTP by random priming. The filters were then washed and subjected to autoradiography.

Neither NRP-154 nor M-NRP1 cells express Bcl-2 RNA (data not shown). In contrast, mRNA levels of Bax, which is pro-apoptotic, are slightly elevated in NRP-154 cells following 24 hours of treatment with TGF-β, but remain unchanged in M-NRP1 cells. However, the basal levels of mRNA expression of the anti-apotpotic factor Bcl-xL are considerably higher in M-NRP1 cells than in NRP-154 cells. Even though TGF-β downregulated expression of Bcl-xL in both cell lines, the mRNA and protein levels of Bcl-xL in M-NRP1 cells after TGF-β treatment remained higher than basal levels in NRP-154 cells. TGF-β dependent elevation of actin mRNA levels was seen in both in NRP-154 and M-NRP1 cells.

These data suggest that elevated levels of Bcl-xL in M-NRP1 cells may contribute to the resistance of these cells to apoptosis.

These data also suggest that aberrant expression and regulation of Bcl-xL in M-NRP1 cells may contribute to the resistance to apoptosis induced by TGF-β. Additionally, it appears that increased levels of Bcl-xL expression in M-NRP1 cells, even following treatment with TGF-β, may contribute, in part, to the resistance of these cells to TGF-β induced apoptosis.

Changes in the regulation of the anti-apoptotic Bcl-2 family member Bcl-xL in the mutant M-NRP1 cells is consistent with other cell types. Reduction in Bcl-xL expression in B-lymphoma cells, mammary epithelial cells, as well as in NRP-154 cells correlates with induction of apoptosis by TGF-β (Saltzman et al., (1998) Exp. Cell Res., 242: 244-254; and Nass et al., (1996) Biochem. Biophys. Res. Commun., 227: 248-256).

B. Western Blot Analysis

NRP-154 and M-NRP1 cell lysates were obtained as described in part A, above. Protein concentrations were determined with the Bradford assay and equal amounts (90 μg) of total cellular protein were loaded on 4-20% gradient gels (Novex), followed by electrophoretic transfer to nitrocellulose membranes (Micron Separations, Inc., Westboro, Mass.). Rabbit anti-Bcl-xL antibodies (Santa Cruz Biotechnology, Inc.) were used at concentrations of 2.5 μg/ml. Immune complexes were detected using the enhanced chemiluminescence (ECL) detection system (Pierce) with a secondary antibody coupled to horseradish peroxidase followed by autoradiography.

### Example VIII

### ARTS

A. ARTS cDNA Isolation

A novel member of the human septin family, called ARTS, for Apoptotic Response TGF-β Signal pathways was isolated. To isolate the cDNA, the Gene Trapper kit (Gibco BRL Life Technologies, Gaithersburg, Md.) was used, following the manufacturer's instructions. A DNA fragment containing the Neomycin resistance gene sequence, which was apart of the retroviral vector used to infect the NRP-154 cells, was used to isolate the mutant clone. 15 mutant clones were found, one of them, M-NRP-1, contining one integration site of the viral vector to its genome, was selected for further investigation. A genomic library was prepared from the rat M-NRP1 cells. Exonic sequences—representing only the expressed RNA sequences—were isolated using RT-PCR from M-NRP1 and were subsequently used to design two primers that were used to screen the human fetal brain cDNA. This screen resulted in the isolation of a 1.8 kb human cDNA fragment, containing the ART Open Reading Frame (ORF).

The DNA fragment containing retroviral vector LTR's is shown in Table 3, below. The exonic sequence is provided in Table 2, below.

TABLE 3

Rat Genomic DNA sequence without viral LTR

(SEQ. ID. NO:6)

GCGGTGGCGG CCGCTCTAGA ATAGTGGATC CTGTATCAGA
TATCTCGCAT
ATCTGATATT TACATTATGG TTCATAACGC TACCAGATTT
ACAGTTCTGA
AGTAGCAACA AAACTAATTC TATGGTTGGG GGATACCACA
ACTTGTGTAA
AAGGACTGCA GCATTAGGAA CATGAGAACC TCTGCTCTAT
TCAGATCAAC
TTAGGCCTCA GCCATCAAAG TATATGCTGT TTCTAAAGGC
AAGTTGCTCC
AAACACCTAG TCTGAAAATA GAAACAAACA AACAAAAAAT
GTGTGTGTAA
TTGGCCACAC ACTGATACCA ATTGTGCTTG TTTTTGATTG
AAATGGACAT
TGATTCTCTT TGGGGCTTGA ATCAAAACTT TTGGCTTTGT
TCACTGACCC
ATAATTGAAA ATAATTTTTT CCTCTGGTCG TGAATTCTTC
TCCCACCCCA
GCCTTGCTCC AGGATTATTA AAATCAAGTC ACATGAAATA
GCACTAATCT
GTCCCGGATG AAATTTTGGA ATTTTTTTTA AATCCTCAAA
CAGCTCAAGT
TTATCTGGGG CTCGGAAGGA ATGAGGAACT ATTAATACCA
CAAGGTTTTC
TTTGAAGGAA GAATAAGCAG GTTGTTTCCT TTTACATGAT
TAGCTTTCAT
CCCTGGGCAC AGCAGCTGGC GGTGGCTGCT TGTCTTTGTT
TTAAACGTTC
TCTTCCTTAT TTCTGTTGGG GAAATGAAAA TGTTAGAAGG
CAAGCATTGT
GGTCATGCCC GAGCTTCAAA CATGGTTCTC AGGAAATGCG
TACAGATAAA
AGGCTTCCTT GCCTTCTACA CTTCAGCCCC TTATGAGACT
CTAAGCCAAT
ATGGTTTGCT CCTTGGGGAA CCTGGGCCCT GGGAGTCTTT
AAATTGCCTG
GAAGGATTTT TAAATTATTT TCATTTTTTT TCTCAAAAAG
CTTCACAGGC
ATCACACATT TCTGAAAATA AAACTGCCTT CATTAACTAG
AATGTCATGG
TATTTTTGCT TTTGATTAGA GTCAAATTAA TTCAGTTTGA
TAACATTGAT
TACAAGCTGA TCTAGAGCTG TGATTAGAGC TTTATATATC
TTAGATGATT
AAATGTGGAA ATAGGTTAAT TATGTCTTAA TAAATGCAGT
TGATTTGATA
GAAAGAAAAT TATACAACCC GAAATTAGGC AGAAAATTAT
GACAAAATTT
CAGAAATTCC TGTATACATC TTGTACGGAG TGGTAAGACC
AATAAAAATT
TCAAATGTATCTATTTTAGAATTCTTCTGGAATCATAAGATTACAAATAT
TTATTACTTT TGGTCACCTT GATATGAAAA
ATACAGAGCCAAATTATTTA
TTCTCTGTCT GCCTGTCTCT CTCTCAATCA
CACACACACACACACACACA
CACACACACACACACACACA
CTGAACTAAACCAAAAACACAATTATACAA
ATTTTATATTGAAAGAACCC

A genomic library prepared from M-NRP1 cells was screened with a 20 bp primer corresponding to the rat exonic sequence. The 20 bp primer is shown in Table 4, below.

TABLE 4

| | |
|---|---|
| First screening primer: | |
| GGTTCTCAGGAAATGCGTACAG | (SEQ. ID. NO:7) |
| Second screening primer: | |
| CATCTTGTACGGAGTGGTAAGACC | (SEQ. ID. NO:8) |

Additionally, several human cDNA libraries were screened with the first screening primer sequence (Table 5). The primer was found to hybridize most strongly with a human fetal brain cDNA library.

The cDNA probe detected two species of approximately 1.8 and 2.0 kb in multiple human tissue blot. The 2 kb band is believed to represent another member of the septin family, showing high homology to ARTS.

B. Sequencing and Characterization of the 1.8 kb cDNA Fragment

Sequencing of the 1.8 kb cDNA fragment revealed an 822 bp open reading frame (Table 6) predicted to encode a polypeptide of 274 amino acids (Table 5). The open reading frame starts at the ATG in position 88 and ends with the stop codon at position 909.

TABLE 6

cDNA Sequence of the ARTS Protein

(SEQ. ID. NO: 1)

GGAATTCCCG GGTCGACCCA CGCGTCCGGT GACGGCGGTG
CTGCGAGGTC GGCGCGCACG TCCGCCGCGG GTCGCTCGGG
CGCTGTCCAG GCGGAGCCGG CCCCGCCCGG GCTGCAGCCA
TGATCAAGCG TTTCCTGGAG GACACCACGG ATGATGGAGA
ACTGAGCAAG TTCGTGAAGG ATTTCTCAGG AAATGCGAGC
TGCCACCCAC CAGAGGCTAA GACCTGGGCA TCCAGGCCCC
AAGTCCCGGA GCCAAGGCCC CAGGCCCCGG ACCTCTATGA
TGATGACCTG GAGTTCAGAC
CCCCCTCGCG GCCCCAGTCC TCTGACAACC AGCAGTACTT
CTGTGCCCCA GCCCCTCTCA GCCCATCTGC CAGGCCCCGC
AGCCCATGGG GCAAGCTTGA TCCCTATGAT TCCTCTGAGG
ATGACAAGGA GTATGTGGGC TTTGCAACCC TCCCCAACCA
AGTCCACCGA AAGTCCGTGA AGAAAGGCTT TGACTTTACC
CTCATGGTGG CAGGAGAGTC TGGCCTGGGC AAATCCACAC
TTGTCAATAG CCTCTTCCTC ACTGATCTGT ACCGGGACCG
GAAACTTCTT GGTGCTGAAG
AGAGGATCAT GCAAACTGTG GAGATCACTA AGCATGCAGT
GGACATAGAA GAGAAGGGTG TGAGGCTGCG GCTCACCATT
GTGGACACAC CAGGTTTTGG GGATGCAGTC AACAACACAG
AGTGCTGGAA GCCTGTGGCA GAATACATTG ATCAGCAGTT
TGAGCAGTAT TTCCGAGACG AGAGTGGCCT GAACCGAAAG
AACATCCAAG ACAACAGGGT GCACTGCTGC CTGTACTTCA
TCTCACCCTT CGGCCATGGG TATGGTCCAA GCCTGAGGCT
CCTGGCACCA CCGGGTGCTG
TCAAGGGAAC AGGCCAAGAG CACCAGGGGC AGGGCTGCCA
CTAGCAGGTG GTCACAGGTT CCTGTTCCCC AGGCTCCGGC
CATTGGATGT TGAATTCATG AAGGCCCTGC ATCAGCGGGT
CAACATCGTG CCTATCCTGG CTAAGGCAGA CACACTGACA
CCTCCCGAAG TGGACCACAA GAAACGCAAA ATCCGGGAGG
AGATTGAGCA TTTTGGAATC AAGATCTATC AATTCCCAGA
CTGTGACTCT GATGAGGATG AGGACTTCAA ATTGCAGGAC
CAAGCCCTAA AGGAAAGCAT
CCCATTTGCA GTAATTGGCA GCAACACTGT AGTAGAGGCC
AGAGGGCGGC GAGTTCGGGG TCGACTCTAC CCCTGGGGCA
TCGTGGAAGT GGAAAACCCA GGGCACTGCG ACTTTGTGAA
GCTGAGGACA ATGCTGGTAC GTACCCACAT GCAGGACCTG
AAGGATGTGA CACGGGAGAC ACATTATGAG AACTACCGGG
CACAGTGCAT CCAGAGCATG ACCCGCCTGG TGGTGAAGGA
ACGGAATCGC AAGTATGACC AGAAGCCAGG ACAAAGCTGG
CAGGGGGAGA TCCCAAGCCT
AGCCTTGGGT GAGACCAAGC CCTACTTTTG TTCTTCTATA
GGCCCTGGGC TCAATCTAAG CGGGTGCTGG GGTCCTCCTC
GCCTTATCAA CCCTTTTCTC CCTTTAGCAA ACTGACTCGG
GAAAGTGGTA CCGACTTCCC CATCCCTGCT GTCCCACCAG
GGACAGATCC AGAAACTGAG AAGCTTATCC GAGAGAAAGA
TGAGGAGCTG CGGCGGATGC AGGAGATGCT ACACAAAATA
CAAAAACAGA TGAAGGAGAA CTATTAACTG GCTTTCAGCC
CTGGATATTT AAATCTCCTC
CTCTTCTTCC TGTCCATGCC GGCCCCTCCC AGCACCAGCT
CTGCTCAGGC CCCTTCAGCT ACTGCCACTT CGCCTTACAT
CCCTGCTGAC TGCCCAGAGA CTCAGAGGAA ATAAAGTTTA
ATAAATCTGT AGGTGGCTAA AAA

*Start and stop codons for the coding sequence (SEQ. ID. NO:3) are underlined and in bold. The first 27 base pairs of the sequence shown in the table are vector sequence and are not included in SEQ. ID. NO: 1. The 5′ UTR for SEQ.ID. NO:1 starts immediately thereafter.

TABLE 7

Amino Acid Sequence of the ARTS Protein

(SEQ. ID. NO:2)

MIKRPLEDTTDDGELSKVKDFSGNASCHPPEAKTWASRPQVPEPRPQAPD
LYDDDLEFRPPSRPQSSDNQQYFCAPAPLSPSARPRSPWGKLDPYDSSED
DKEYVGFATLPNQVHRKSVKKGFDFTLMVAGESGLGKSTLVNSLFLTDLY
RDRKLLGAEERIMQTVEITKHAVDIEEKGVRLRLTIVDTPGFGDAVNNTE
CWKPVAEYIDQQFEQYFRDESGLNRKNIQDNRVHCCLYFISPFGHGYGPS
LRLLAPPGAVKGTGQEHQGQGCH

As shown in the amino acid sequence in Table 7, ARTS contains a phosphate binding site P-loop motif (shown in bold italics). Generally, the consensus sequence of a P-loop motif can be represented by the following: GXSGXGKS/T (SEQ. ID. NO: 4) (Aravind et al., (1999) Trends Biochem. Sci. 24: 47; and Saraste et al., (1990) Trends Biochem. Sci. 11: 430). P-loop GTP-binding motifs are conserved in the septin family of proteins and found in many ATP/GTPases, including CED-4 (Chaudhary et al., (1998) J. Biol. Chem. 273: 17708) and Apaf-1 (Hu et al., (1999) EMBO J. 18; 3586), which are major regulators of apoptosis. FIG. 5 shows the alignment of the P-loop domains of the apoptogenic proteins CED-4 and Apaf-1 and septin family members (human) ARTS and NEDD5, Drosophila PEANUT, C. elegans C-CDC10 and S. cerevisiae Y-CDC10.

A BLAST search of Genbank the 822 bp open reading frame sequence showed that ARTS matches human chromosome 17q22-23 genomic sequences (gb HRPK AC005666). It is believed that ARTS is the product of an alternatively spliced form of the hH5/PNUTL2/hCD-Crel-2 septin family gene (Xie et al., (1997) Cell Motil. Cytoskeleton 43: 52; McKie et al., (1999) Hum. Genet. 101: 6; and Zieger et al., (1997) J. Clin. Invest. 99: 620).

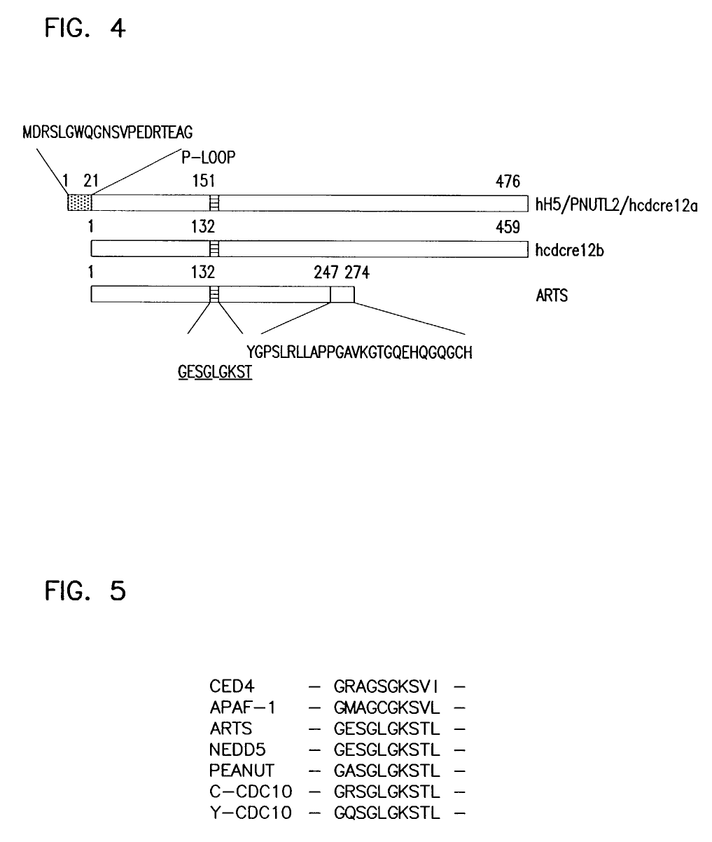

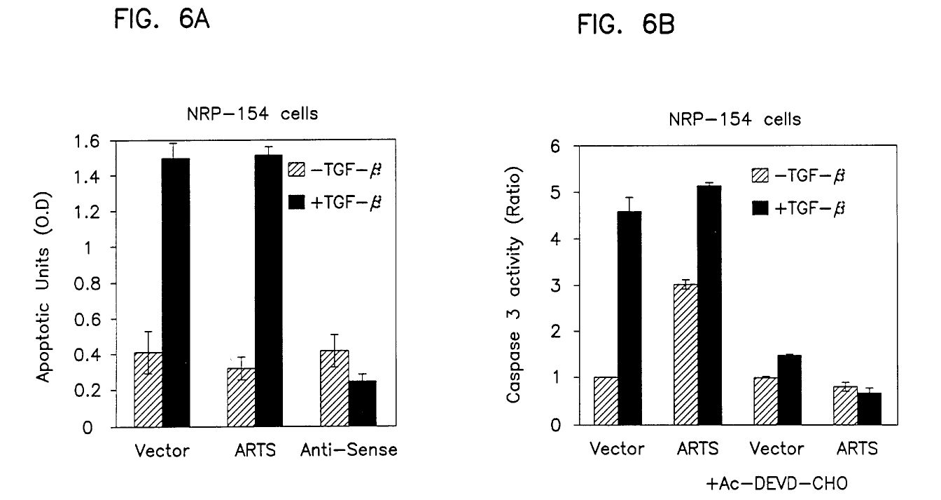

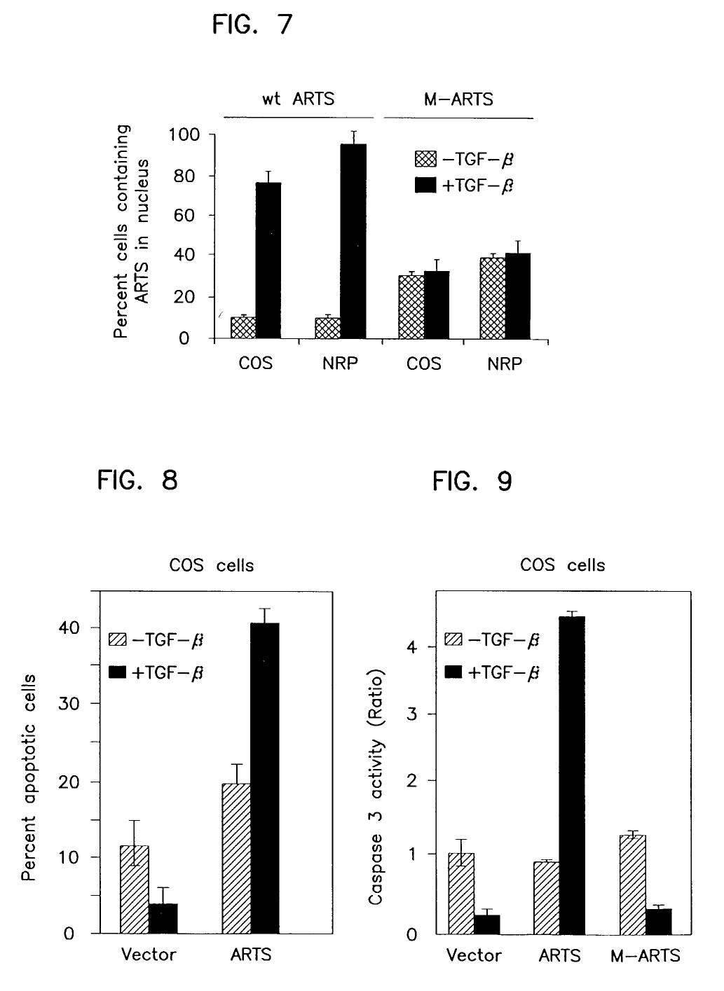

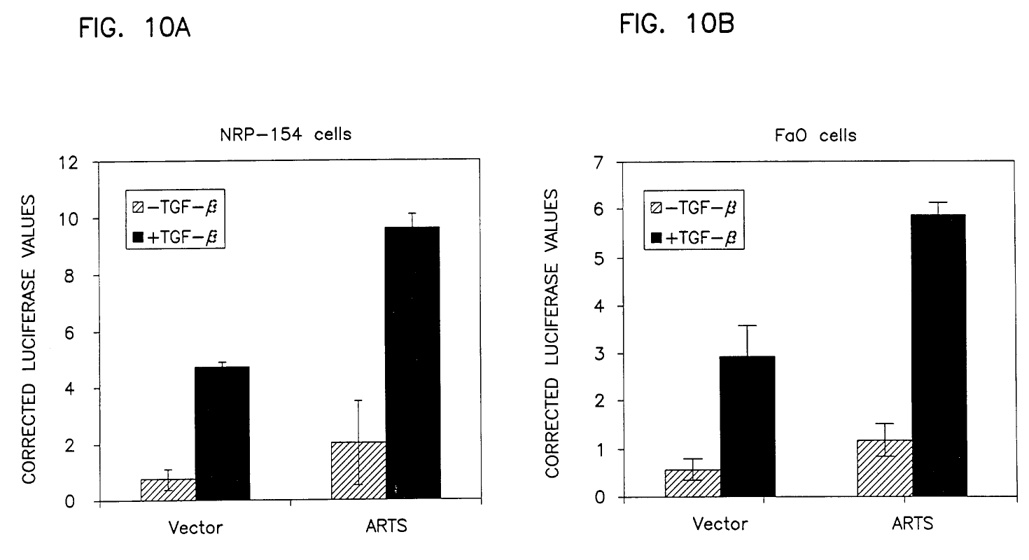

ARTS shares its N-terminal start site with hCDCrel-2b and has a unique 27 amino acid C-terminus (GPSLRLLAPPGAVKGTGQEHQGQGCH; SEQ. ID. NO: 5) not found in any of the other products of this gene. FIG. 4 shows the alignment of the sequences encoding hH5/PNUTL2/hcdcrel2a, hcdcrel2b, and ARTS, showing the conserved P-loop and the unique 27 amino acid C-terminus of ARTS.

C. Expression of ARTS

Expression of the ARTS protein was examined to determine whether its expression is altered in cells that are resistant to apoptosis induced by TGF-β (e.g., M-NRP1 cells).

Briefly, NRP-154, M-NRP1 and COS cells (2×10⁶) were plated in 10-cm Falcon tissue culture dishes with 10 ml DMEM/F12 or DMEM for COS cells, containing 15 mM HEPES and 10% calf serum. Cell lysates were prepared from confluent cell cultures with and without treatment with TGF-β (10 ng/ml) for 24 hours. Briefly, cells were rinsed with phosphate buffered saline, scraped into 0.5 ml of ice-cold lysis buffer (phosphate buffered saline, 1% Nonidet P40, 0.5% sodium deoxycholate, 0.1% SDS) containing protease and phosphatase inhibitors (50 μg/ml phenylmethylsulfonyl fluoride, 30 μg/ml aprotonin, 5 μg/ml leupeptin, 100 mM sodium orthovanadate), and passed several times through a 21 gauge needle.

The expression of endogenous ARTS in the cell lysates from NRP-154, M-NRP1 and COS cells was detected by a Western blot assay. Rabbit anti-ARTS polyclonal antibodies were raised to the unique 27 amino acid C-terminus of ARTS (custom, Sigma, Israel). For Western blotting, the anti-ARTS antibody was used at a concentration of 2.5-5 μg/ml. Ponceau S staining (Sigma) was used as a loading control.

ARTS expression is elevated in lysates of M-NRP1 cells compared to NRP-154 cells. COS cells express3e about the same amount of endogenous ARTS as NRP-54.

D. Molecular Weight of ARTS

The molecular weight of the ARTS protein was determined in lysates of metabolically labeled COS cells, NRP-154 cells, and A549 human lung carcinoma cells.

The AU5-ARTS was generated by subcloning a 0.822-kb fragment containing the entire ARTS coding sequence into the Bgl II-Eco RI sites of pCEF-AU5 vector. Anti-AU5 antibodies purchased from “Babco, Richmond, Calif.”. The cells were cultured and lysed as described above (C). Lysates were clarified at 100,000×g in an ultracentrifuge, precleared with protein G-Sepharose (Pharmacia Biotech Inc.) and normal rabbit serum.

The cells were then transfected with the ARTS-AU5 tagged construct using Lipofectamine (Gibco BRL Life Technologies, Gaithersburg, Md.) or FuGENE 6 (Boehringer-Mannheim) according to the manufacturer's protocol. Cell lysates were prepared as described in Part C, above. T protein was immunoprecipitated with 10 μg/ml of anti-AU5 antibody for 2 h. Complexes were precipitated with protein G-Sepharose and washed in high salt precipitation buffer (1 M NaCl, 50 mM Tris, pH 7.5, 0.1% SDS, 0.5% deoxycholate, protease, and phosphatase inhibitors), and the resulting immunoprecipitates were resolved by SDS-polyacrylamide gel electrophoresis on 4-20% gels and visualized by Autoradiography.

Endogenous ARTS was detected in all three cell types as approximately a 34 k band. The 34 kDa molecular weight is slightly higher than its predicted molecular weight, suggesting post-translational modifications of ARTS.

E. Intracellular Distribution of ARTS

The intracellular distribution of ARTS was examined by immunohistochemical staining of NPR-154 and M-NRP1 cells with anti-ARTS antibody.

Briefly, 2×10⁴ cells were seeded in chamber slides (Nunc) and transfected with suitable DNA constructs after 24 hours. After an additional 24 hours, cell media was changed to include 1% fetal calf serum with or without 10 ng/ml TGF-β.

The cells were then fixed with 4% paraformaldehyde and 5% sucrocse in PBS for 20 minutes, washed, and treated with 0.5% tritonX100 in PBS for 5 minutes. The cells were then washed with PBS and blocked with 5% BSA in PBS for 30 minutes. Cells were incubated with 2.5-5 μg/ml rabbit anti-ARTS (custom, Sigma Israel) antibodies raised to the 27 aa unique C-terminus of ARTS. Subsequently, the cells were combined with biotinylated secondary antibodies and streptavidin-HRP (peroxidase). The reaction was developed with Aminoethyl Carbazole (ARC).

Immunohistochemical staining indicates that the ARTS protein is localized predominantly to the cytoplasm of NRP-154 cells. In contrast, the ARTS protein is localized in the nucleus of M-NRP1 cells, rather than the cytoplasm.

The overexpression and mislocalization of ARTS suggests that the protein has been mutated in such a way as to block the apoptotic activity.

F. ARTS Activity

In this experiment, the activity of ARTS was examined. Specifically, whether ARTS acts downstream of the TGF-β receptors was examined.

NRP-154 and FaO cells were seeded to 50% confluence in six-well plates in DMEM-F12 or DMEM, respectively, containing 10% fetal calf serum overnight.

Antisense- and sense-ARTS were generated by PCR using ARTS cDNA containing the 5′UTR of ARTS as a template. The PCR fragments were subcloned into the Bam H1-Eco R1 sites of pcDNA3 vector containing the Pcmv promotor (invitrogen, Carlsbad, Calif.).

NRP-154 cells and FaO cells were transfected with lug/sample p3TP-Lux reporter (a gift from Dr. Joan Massague), which contains 3 repeats of an AP-1 site from the collagenase promoter linked to a short region of the PAI-1 promoter along with equal concentrations of either pcDNA3 control vector (PGL2 vector commercially available from Promega, Madison, Wis.) or the ARTS-pcDNA3 construct, described above.

The cells were then treated with 10 ng/ml TGF-β for 24 hours.

After 24 hours, a cell lysate was prepared essentially as described in Part C, above.

Luciferase activity was determined in the cell lysate using an assay kit (BD Pharmingen, San Diego, Calif.), and a MLX Microtiter plate luminometer (Dynex Technologies, Chantilly, Va.). 1 ug/sample pSV-galactosidase (beta galactosidase under control of simian virus 40 promoter) was transfected in each sample, and levels of beta galactosidase activity were used to determine level of transfection in each individual sample and normalize the 3TP-Lux counts accordingly.

Experiments were repeated three times with similar results. The results are expressed as the mean (±SEM) of duplicate luciferase assay, corrected for transfection efficiency.

In NRP-154 cells and FaO rat hepatoma cells, which respond to TGF-β by apoptotic death (Hsing et al., (1996) Cancer Res. 56: 5146; and Choi et al., (1998) Hepatology 27: 415) both basal and TGF-β-induced luciferase activity were increased by cotransfection of cells with ARTS (FIGS. 10A,B). Thus, it appears that ARTS activates the TGF-β responsive 3TP-Lux promoter construct in cells sensitive to TGF-β-induced apoptosis.

PGLP2, containing only the luciferase gene driven by a CMV promoter, showed no response to either ARTS transfection or TGF-β treatment of cells (data not shown). These results demonstrate that ARTS sensitizes cells to TGF-β, but suggest that its primary action might be on TGF-β signaling pathways other than those which mediate activation of the 3TP-Lux promoter.

G. Effect of Transfected wt ARTS on Appoptosis

To determine whether ARTS plays a role in TGF-β induced apoptosis, wild type ARTS was was transiently transfected into NRP-154 cells and M-NRP1.

Cells were seeded to 50% confluence in six-well plates in DMEM-F12 containing 10% fetal calf serum overnight.

ARTS whole cDNA in PCMV-sport vector was isolated from a human fetal cDNA library in the pcmv-sport vector (Gibco BRL Life Technologies). This vector is different from AU5 tagged vector.

The NRP-154 and M-NRP1 cells were cotransfected with a total of 2 μDNA, 1 μg ARTS-pcmv-sport and 1 μg a GFP vector (Clontech, Palo-Alto, Calif.), a marker for transfected cells, using Lipofectamine (Gibco BRL Life Technologies, Gaithersburg, Md.) or FuGENE 6 (Boehringer-Mannheim) according to the manufacturer's protocol.

After 24 hours, cells media was changed to contain 1% fetal calf serum with or without 10 ng/ml TGF-β for an additional 24 hours.

GFP-positive cells were sorted using FACS.

The sorted population was stained with streptavidin-sulforhodamin.

The number of apoptotic cells was measured using the TACS Annexin-V-biotin (Trevigen, Gaithersburg, Md.).

Transfection of M-NRP1 cells with wildtype ARTS restored, in part, the sensitivity of these cells to TGF-β induced apoptosis. These data suggest that ARTS is altered in M-NRP1 cells in such a way that its apopotgenis activity has been compromised. Since transfection with human ARTS partially restored an apoptotic response to TGF-β in M-NRP1 cells, it seems reasonable to conclude that other components of the ARTS apoptotic pathway remain functional in the mutant cells.

1. TUNEL

COS cells were cultured as previously described (Section E, above). COS cells were selected because they do not undergo apoptosis with TGF-β treatment alone. When these cells are transfected with ARTS and treatment with TGF-β they are induced to cell death.

COS cells were then transfected with control vector or wildtype ARTS, essentially as described above, and treated with 10 ng/ml TGF-β for 24 hours.

The apoptotic response of COS cells to TGF-β was assessed by a TUNEL assay (Boehringer-Ingelheim, now Roche, Indianapolis, Ind.) following the manufacturer's instructions.

COS cells transfected with control vector did not undergo apoptosis when treated with TGF-β. In contrast, cells transfected with ARTS exhibited strong TUNEL staining following treatment with TGF-β. Conclusion: ARTS overexpression in COS cells enables them to respond in apoptotic death to TGF-β stimulation.

2. Indirect Immunofluorescence

COS cells were cultured as desribed previously (Section E, above) and cotransfected with either empty empty AU5 vector (based on pcDNA3 vectors provided by Dr. Silvio Gukind) or ARTS-AU5 with X-vector. ARTS-AU5 was prepared as desribed above. X-vector is a general term to describe any other vector which was cotransfected together with ARTS to asses its effect on ARTS activity.

24 hours after transfection, cell media was changed to contain 1% fetal calf serum with or without 10 ng/ml TGF-β and the cells were incubated for another 24 hours. The cells were then washed twice with PBS and fixed with 4% paraformaldehyde in PBS. Cells were blocked with 5% BSA in PBS for 30 minutes, then incubated with anti-AU5 fluroescein antibodies (Babco, Richmond, Calif.). The cells were also counterstained with DAPI for counting of fragmented nuclei.

The percentage of cells exhibiting apoptotic nuclear fragmentation was determined, and counted, compared to the transfected population.

The cells transfected with empty vector and wt ARTS responded identically. This indicates that ARTS overexpression in COS cells by itself does not induce apoptosis. The apoptotic cell death in COS cells trasnfected with ARTS is ligand dependent, and induced with the addition of TGF-β. The results are expressed as the mean (±SEM) of duplicate assays, and repeated three times with similar results.

Both an increased basal level and TGF-β-induced level of apoptosis were found in cells transfected with the ARTS expression construct as compared to vector only transfectants. This suggests that overexpresion of ARTS may shift the balance of TGF-β signaling pathways towards apoptotic endpoints in certain cells such as COS and A549 (data not shown) that do not normally undergo apoptosis in response to TGF-β.

H. Effect of Endogenous ARTS on Apoptosis

To determine whether endogenous arts is essential for TGF-β induced apoptosis, an anti-sense construct of ARTS was transfected into NRP-154 cells.

NRP-154 cells were cultured as described above. The cells ere transfected with anti-sense ARTS using Lipofectamine (Gibco BRL Life Technologies, Gaithersburg, Md.) or FuGENE 6 (Boehringer-Mannheim) according to the manufacturer's protocol.

Cell lysates were prepared from confluent cell cultures, with and without treatment with TGF-β (10 ng/ml) for 24 hours. Briefly, cells were rinsed with phosphate buffered saline, scraped into 0.5 ml of ice-cold lysis buffer (phosphate buffered saline, 1% Nonidet P-40, 0.5% sodium deoxycholate, 0.1% SDS) containing protease and phosphatase inhibitors (50 μg/ml phenylmethylsulfonyl fluoride, 30 μg/ml aprotinin, 5 μg/ml leupeptin, 30 100 mM sodium orthovanadate), and passed several times through a 21-gauge needle.

Endogenous protein levels were measured by immunohistochemistry or Western blot analysis of cell lysates. Internucleosomal fragmentation of DNA was quantified by TUNEL and an Elisa assay.

a. Immunohistochemistry

The effect of anti-sense ARTS on expression of endogenous ARTS in NRP-154 cells was assessed by immunohistochemical techniques, essentially as described above under “Indirect Immunofluorescence.” After fixation, the cell lysates were stained using Histostain-Plus kit (Zymed, S. San Francisco, Calif.). The slides were developed with aminoethyl carbazole (AEC) substrate solution (Zymed, S. San Francisco, Calif.).

b. Western Blot Analysis

The effect of anti-sense ARTS on expression of endogenous ARTS in NRP-154 cells was confirmed by Western Blot analysis.

Cell lysates were prepared from confluent cultures with and without treatment with TGF-β (10 ng/ml) for 48 hours. Briefly, cells were rinsed with phosphate buffered saline, scraped into 0.5 ml of ice-cold lysis buffer (phosphate buffered saline, 1% Nonidet P40, 0.5% sodium deoxycholate, 0.1% SDS) containing protease and phosphatase inhibitors (50 μg/ml phenylmethylsulfonyl fluoride, 30 μg/ml aprotinin, 5 μg/ml leupeptin, 30 100 mM sodium orthovanadate), and passed several times through a 21-gauge needle. Cell lysates were then incubated on ice for 30 min and microfuged for 20 min at 4° C. Protein concentrations were determined with the Bradford assay. Equal amounts (90 ug) of total cellular protein were loaded on 4-20% gradient gels (Novex), followed by electrophoretic transfer to nitrocellulose membranes (Micron Separations, Inc., Westboro, Mass.). Anti-ARTS antibodies, (costum, Sigma, Israel) were used at concentration of 2.5 μg/ml. Immune complexes were detected using the enhanced chemiluminescence (ECL) detection system (Pierce, Rockford, Ill.) with a secondary antibody coupled to horseradish peroxidase followed by autoradiography.

c. TUNEL

Functional effects of antisense ARTS in NRP-154 cells were determined using the TUNEL assay.

Briefly, cells were cultured as previously described. 24 hours after transfection, cells media was changed to contain 1% fetal calf serum with or without 10 ng/ml TGF-β for an additional 24 hours. Cells were then fixed and stained using the In Situ Cell Death Detection—POD kit (Roche, Indianapolis, Ind.) according to manufacture's instructions, and developed with Aminoethyl Carbazole (AEC) substrate solution (Zymed, S. San Francisco, Calif.).

d. Elisa

Functional effects of antisense ARTS in NRP-154 cells were determined using a cell death detection Elisa assay (Boehringer-Mannheim).

Cell lysates were prepared from confluent cultures with and without treatment with TGF-β (10 ng/ml) for 48 hours. Briefly, cells were rinsed with phosphate buffered saline, and lysed using lysis buffer provided with Cell death detection Elisa kit (Roche, Indianapolis, Ind.) according to the manufacturers' protocols. Results were read using FL-600 microplate fluorescence reader (Bio-Tek).

e. Results

NRP-154 cells transfected with anti-sense ARTS exhibited marked reduction in their apoptotic response to TGF-β. This suggests that expression of endogenous ARTS is necessary to direct TGF-β signaling towards apoptotic endpoints.

I. Apoptotic Pathway

Caspase 3 is activated by a proteolytic cascade in cells undergoing apoptosis (Núñez et al., (1998) Oncogene 17: 3237) and is a key player in programmed cell death. To investigate whether ARTS-dependent apoptotic pathway induced by TGF-β involves effector caspases, Caspase 3-like activity was assayed in COS cells and NRP-154 cells.

Cells were seeded to 50% confluence in six-well plates in media containing 10% fetal calf serum overnight. Following 24 hours after transfection, cells media was changed to contain 1% fetal calf serum with or without 10 ng/ml TGF-β for additional 24 hours. Cells were treated with caspase 3 aldehyde inhibitor (Ac-DEVD-CHO) supplied with caspase 3 fluorescent activity kit according to the manufacturers' protocols (Pharmingen, San-Diego, Calif.). Results were read using FL-600 microplate fluorescence reader (Bio-Tek).

In COS cells, which do not undergo apoptosis in response to TGF-β, overexpression of ARTS (due to transfection of an expression vector that contains the ARTS ORF) caused a marked increase in the activation of caspase 3 following treatment with TGF-β. This is consistent with the ability of ARTS to increase TGF-β dependent apoptosis in COS cells.

Similar data to those described for COS cells, were found for A549 cells. This indicates that, in A549 cells, like COS cells, which do not undergo apoptosis in response to TGF-β, overexpression of ARTS caused a marked increase in caspase 3 activity following TGF-β treatment.

In contrast, in NRP-154 cells, which are sensitive to TGF-β induced apoptosis, caspase 3 was activated by treatment with TGF-β. Overexpression of ARTS had no further effect on this activity. This is similar to the lack of effect of ARTS on TGF-β induced apoptosis.

Treatment with caspase 3 aldehyde inhibitor Ac-DEVD-CHO inbitited the TGF-β induced caspase activity in all cells examined.

Data are standardized as fold-changes relative to untreated vector alone.

These results indiate that ARTS is essential for TGF-β induced apoptosis, and involves activation of caspase 3. This suggests that apoptosis induced by TGF-β and mediated by ARTS likely uses previously described apoptotic pathways that converge on activation of caspase-3 like effector cysteine proteases.

J. P-Loop GTP-Binding Motif

To determine the role of the P-loop GTP-binding motif in the apoptotic activity of ARTS, a mutant construct of ARTS (M-ARTS) was transfected into COS cells.

M-ARTS, a mutant construct containing three mutations in the P-loop GTP-binding motif (GKS→ENP) was prepared. Briefly, point mutations in the P-loop GTP-binding motif were introduced using the QuikChange™ Site-Directed mutagenesis kit (Stratagene, La Jolla, Calif.), according to the manufacturer's instructions. Two complementary oligonucleotides encompassing the P-Loop domain were prepared. One of the oligonucleotides is shown below:

5′-GGAGAGTCTGGCCTGGAGAATCCCACACTTGTCAATAGCC-3′ (SEQ. ID. NO: 9) (changed nucleotides are underlined).

The M-ARTS construct was prepared by cloning the mutant sequence into pCEF-AU5 vector (Bam HI-Eco RI sites).

COS cells were cultured and transfected, as described above, with equal concentrations of either pcDNA3 control vector, ARTS or M-ARTS-pcDNA3 using the Lipofectamine transfection reagent (Gibco BRL Life Technologies, Gaithersburg, Md.). The cells were treated with 1 o ng/ml TGF-β for 24 hours.

1. TUNEL

Apoptosis was examined in a TUNEL assay (Boehringer-Mannheim), according to the manufacturer's instructions.

Unlike ARTS, M-ARTS failed to induce competence to undergo apoptosis in the presence of TGF-β. This suggests that the P-loop motif is critical in the apoptogenic activity of ARTS.

2. Caspase 3

Caspase 3 activity was monitored as described above.

3. Conclusion

Mutation of the P-loop domain appears to abrogate ARTS' ability both to translocate to the nucleus and to induce apoptosis. TGF-β dependent apoptosis requires an intact P-loop motif.

K. Translocation of ARTS

The localization of ARTS, in the presence or absence of TGF-β, was examined by an immunofluorescence assay.

NRP-154 and COS cells were cultured and transfected as described above, and transfected with an ARTS-AU5 tagged construct, essentially as described in Part D, above. The cells were then treated with 10 ng/ml TGF-β.

An immunofluorescence assay was performed essentially as described in Part G2, above using anti-AU5 antibodies (commercially available Babco, Richmond, Calif.). Anti-AU5 FITC-conjugated secondary antibodies (Pierce) were used to detect transfected ARTS.

A cystostolic particulate pattern of staining for ARTS was observed, similar to that of untransfected NRP-154 cells stained with an anti-ARTS antibody. Further examination of this intracellular localization by confocal and immunoelectron microscopy showed that ARTS staining was localized to mitochondria and appeared to be clustered in protein-rich areas. Confocal images were obtained using a confocal microscope (Bio-Rad MRC-1024 scanhead attached to a Zeiss Axiovert 135M). Immunoelectron microscopy was formed as described by Wang et al., (1998), Endocrinology 139: 3903.

Following TGF-β treatment, ARTS-AU5 translocates to from the mitochondria to the nucleus, both in COS and NRP-154 cells causing fragmentation of nuclei in the transfected cells. Nuclear fragmentation was observed by staining the cells wth DAPI. A similar pattern is seen in non-transfected NRP-154 cells, where endogenous ARTS can be seen in the degraded nucleus following treatment with TGF-β.

Immuno-gold electron microscopy of ARTS-AU5 transfected COS cells demonstrates distinct locatilzation of ARTS in mitochondria and electron-dense lysosomal-like bodies.

In contrast, ARTS is localized principally in the nucleus in M-NRP1 cells, indpendent of TGF-β treatment (data not shown).

M-ARTS is unable to translocate to the nucleus in response to treatment of cells with TGF-β, leaving the nuclei of transfected cells intact, consistent with it inability to inactivate capsase 3 and to induce apoptosis in COS cells. Thus, translocation of ARTS from the mitochondria to the nucleus requires and intact P-loop motif.

An N-terminal deletion mutant of ARTS (81 amino acids shorter than ARTS) was generated by PCR using ARTS cDNA. The DNA was subcloned into the BGI II-EcoRI sites of pCEF-AU5 vector. is unable to localize to mitochondria or to translocate to nuclei. In contrast, C-terminal truncation results in constitutive nuclear localization of ARTS. This suggests that mitochondrial processing might play a role in the nuclear localization of ARTS.

A control transfection with steroidogenic acute regulatory protein, StAR34 shows a homogenous distribution of StAR in the mitochondria distinct from that of ARTS. StAR34 is a typical mitochontrial protein.

Following TGF-β treatment, ARTS translocates to the nucleus causing nuclear fragmentation as seen with DAPI stain.

The invention has been described with reference to various specific and preferred embodiments and techniques. However, it should be understood that many variations and modifications may be made while remaining within the spirit and scope of the invention. The disclosures of all references cited herein are hereby incorporated by reference in their entirety.

What is claimed is:

1. An isolated nucleic acid sequence comprising the sequence of SEQ. ID. NO: 1, or its complement.

2. An isolated nucleic acid sequence encoding the amino acid sequence shown in SEQ. ID. NO: 2.

3. A hybridization probe comprising at least 15 consecutive nucleic acids of SEQ. ID. NO: 1, or its complement.

4. A hybridization probe comprising at least 15 consecutive nucleic acids having at least 85% homology to SEQ. ID. NO: 1, or its complement.

5. An isolated nucleic acid comprising a sequence that encodes a polypeptide with the amino acid sequence of SEQ. ID. NO: 2.

6. An isolated nucleic acid sequence comprising a sequence that hybridizes under stringent conditions to the nucleic acid sequence of SEQ. ID. NO: 1, or the complement of SEQ. ID. NO: 1.

7. An isolated nucleic acid sequence comprising a fragment of SEQ. ID. NO: 3 of at least 15 consecutive nucleic acids.

8. An isolated nucleic acid sequence comprising a fragment of SEQ. ID. NO: 1, comprising a P-Loop Domain consensus sequence.

9. An isolated nucleic acid sequence comprising a variant of SEQ. ID. NO: 1, comprising at least SEQ. ID. NO: 5.

10. An isolated nucleic acid sequence comprising a sequence at least 80% identical to SEQ. ID. NO: 3, or its complement.

11. An isolated protein comprising the amino acid sequence shown in Table 2.

12. An isolated nucleic acid sequence comprising a sequence that encodes a polypeptide having an amino acid sequence that is at least 80% identical to SEQ. ID. NO: 2.

13. An expression vector comprising the coding sequence of the nucleic acid sequence of claims 1, 2, or 5-12, operably linked to an expression control sequence.

14. A cultured cell comprising the vector of claim 13.

15. A cultured cell transfected with the vector of claim 13, or a progeny of said cell, wherein the cell expresses a polypeptide encoded by the expression vector of claim 13.

16. An antisense oligonucleotide that inhibits ARTS protein expression, said oligonucleotide binding to ARTS-encoding RNA, wherein said antisense compound specifically hybridizes with and inhibits expression of ARTS.

17. A method for inhibiting expression of ARTS protein in a cell, comprising administering to the cell an effective amount of the antisense oligonucleotide of claim 16.

18. A method for increasing sensitivity of a cell to apoptosis, comprising introducing into the cell an effective amount of the nucleic acid of claims 1, 2 or 5-12.

19. The method of claim 18, wherein the cell is induced to apoptosis by TGF-β or a chemotherapeutic agent.

20. A method for increasing sensitivigy of a cell to apoptosis, comprising introducing into the cell an effective amount of ARTS protein.

21. A method for reducing the sensitivity of a cell to apoptosis, wherein the cell is induced to apoptosis by TGF-β, comprising introducing into the cell an effective amount of the antisense oligonucleotide of claim 16.

22. A method for detecting apoptosis of a cell, comprising determining the presence of the nucleic acid sequence of claim 1 within the cell.

23. A method for detecting apoptosis of a cell, comprising determining the presence of a polypeptide encoded by the nucleic acid sequence of claim 1 within the cell.

24. A method for detecting apoptosis of a cell, comprising determining a location of the nucleic acid sequence of claim 1 within the cell.

25. A method for detecting apoptosis of a cell, comprising determining a location of a polypeptide encoded by the nucleic acid sequence of claim 1 within the cell.

26. An antibody that specifically binds a polypeptide encoded by nucleic acid sequence of claim 1.

27. A kit for detection of ARTS protein, comprising the antibody of claim 26.

28. A kit for detection of a nucleic acid sequence encoding ARTS protein, comprising the nucleic acid sequence of claim 1, 2 or 5-12.

29. A single stranded oligonucleotide comprising SEQ. ID. NO: 3, or the complement thereof.

30. A method for producing ARTS protein, comprising transfecting a cell with the vector of claim 13 and expressing ARTS protein in the cell.

31. A method of detecting a nucleic acid encoding ARTS in a sample, comprising introducing into the sample the nucleic acid sequence of claim 1, 2 or 5-12.

32. The method of claim 31, wherein the sample comprises at least one cell.

33. The method of claim 31, wherein the nucleic acid sequence comprises a label.

34. A method of detecting ARTS protein in a sample, comprising introducing into the sample an anti-ARTS antibody.

35. The method of claim 34, wherein the sample comprises at least one cell.

36. The method of of claim 34, wherein the sample comprises cell lysate.

37. The method of claim 34, wherein the anti-ARTS antibody comprises a label.
